SEQUENCE LISTING (60 sequences)

SEQ ID NO: 1 - protein, 6 aa, synthetic construct
  REGION 1..6: note = Flexible linker 2 - (GSG)2
GSGGSG

SEQ ID NO: 2 - protein, 9 aa, synthetic construct
  REGION 1..9: note = Flexible linker 3 - (GSG)3
GSGGSGGSG

SEQ ID NO: 3 - protein, 5 aa, synthetic construct
  REGION 1..5: note = Flexible linker 4 - (G4S)1
GGGGS

SEQ ID NO: 4 - protein, 15 aa, synthetic construct
  REGION 1..15: note = Flexible linker 5 - (G4S)3
GGGGSGGGGSGGGGS

SEQ ID NO: 5 - protein, 20 aa, synthetic construct
  REGION 1..20: note = Flexible linker 6 - (G4S)4
GGGGSGGGGSGGGGSGGGGS

SEQ ID NO: 6 - protein, 12 aa, synthetic construct
  REGION 1..12: note = Flexible linker 7
GSAGSAAGSGEF

SEQ ID NO: 7 - protein, 18 aa, synthetic construct
  REGION 1..18: note = Flexible linker 8
KESGSVSSEQLAQFRSLD

SEQ ID NO: 8 - protein, 14 aa, synthetic construct
  REGION 1..14: note = Flexible linker 9
EGKSSGSGSESKST

SEQ ID NO: 9 - protein, 5 aa, synthetic construct
  REGION 1..5: note = Rigid linker 1
EAAAK

SEQ ID NO: 10 - protein, 15 aa, synthetic construct
  REGION 1..15: note = Rigid linker 2 - (EAAAK)3
EAAAKEAAAKEAAAK

SEQ ID NO: 11 - protein, 14 aa, synthetic construct
  REGION 1..14: note = Rigid linker 3 - (AP)7
APAPAPAPAPAPAP

SEQ ID NO: 12 - DNA, 2235 bp, synthetic construct
  misc_feature 1..2235: note = gH-SpyTag-His
  misc_feature 1146: note = C>A mutation at position 1146
  sig_peptide 1..69
  misc_feature 70..2151: note = Ectodomain
  misc_feature 2152..2157: note = Transmembrane domain (truncated)
  misc_feature 2158..2175: note = Linker
  misc_feature 2176..2214: note = SpyTag
  misc_feature 2215..2232: note = 6x His tag
  misc_feature 2233..2235: note = Stop codon
  source 1..2235: mol_type = other DNA
atgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacatgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcggaagcggaggctctggtgcccatatcgtgatggtggacgcctacaagcctaccaaacatcatcaccatcaccactaa

SEQ ID NO: 13 - DNA, 2157 bp, synthetic construct
  misc_feature 1..2157: note = gH with truncated transmembrane domain
  misc_feature 1146: note = C>A mutation at position 1146
  sig_peptide 1..69
  misc_feature 70..2151: note = Ectodomain
  misc_feature 2152..2157: note = Transmembrane domain (truncated)
  source 1..2157: mol_type = other DNA
atgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacatgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctc

SEQ ID NO: 14 - DNA, 18 bp, synthetic construct
  misc_feature 1..18: note = Linker from gH construct
  source 1..18: mol_type = other DNA
ggaagcggaggctctggt

SEQ ID NO: 15 - DNA, 39 bp, synthetic construct
  misc_feature 1..39: note = Spytag
  source 1..39: mol_type = other DNA
gcccatatcgtgatggtggacgcctacaagcctaccaaa

SEQ ID NO: 16 - DNA, 837 bp, Human cytomegalovirus
  sig_peptide 1..90
  misc_feature 91..834: note = Ectodomain
  misc_feature 835..837: note = Stop codon
  source 1..837: mol_type = other DNA
atgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctaa

SEQ ID NO: 17 - DNA, 702 bp, synthetic construct
  misc_feature 1..702: note = UL130 - C-tag
  sig_peptide 1..75
  misc_feature 76..642: note = Ectodomain
  misc_feature 643..687: note = Linker
  misc_feature 688..699: note = C-tag
  misc_feature 700..702: note = Stop codon
  source 1..702: mol_type = other DNA
atgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttggaggcggaggatctggcggaggtggaagtggcggaggcggatctgagcccgaggcctaa

SEQ ID NO: 18 - DNA, 642 bp, synthetic construct
  misc_feature 1..642: note = UL130 (signal sequence and ectodomain)
  sig_peptide 1..75
  misc_feature 76..642: note = Ectodomain
  source 1..642: mol_type = other DNA
atgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtt

SEQ ID NO: 19 - DNA, 45 bp, synthetic construct
  misc_feature 1..45: note = Linker from UL130 construct
  source 1..45: mol_type = other DNA
ggaggcggaggatctggcggaggtggaagtggcggaggcggatct

SEQ ID NO: 20 - DNA, 759 bp, Human cytomegalovirus
  misc_feature 634: note = T>C mutation at position 634
  sig_peptide 1..81
  misc_feature 82..164: note = Ectodomain
  intron 165..287
  misc_feature 288..422: note = Ectodomain
  intron 423..542
  misc_feature 543..756: note = Ectodomain
  misc_feature 757..759: note = Stop codon
  source 1..759: mol_type = other DNA
atgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgtaagtctcttcctgagggccttacagcctatgggagagtaagacagagagggacaaaacatcattaaaaaaaaaagtctaatttcacgttttgtaccccccttcccctccgtgttgtaggttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagtga

SEQ ID NO: 21 - DNA, 498 bp, Human cytomegalovirus
  sig_peptide 1..54
  misc_feature 55..236: note = Ectodomain
  intron 237..344
  misc_feature 345..495: note = Ectodomain
  misc_feature 496..498: note = Stop codon
  source 1..498: mol_type = other DNA
atgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagaggtgagggtacgcgctaaagatgcatgacaacgggaaggtaagggcgaacgggtaacgggtaagtaaccgcatggggtatgaaatgacgttcggaacctgtgcttgcagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactag

SEQ ID NO: 22 - DNA, 1008 bp, synthetic construct
  misc_feature 1..1008: note = SpyCatcher-HBsAg
  misc_feature 1..276: note = SpyCatcherDeltaN1
  misc_feature 277..303: note = Flexible Linker
  misc_feature 304..315: note = PVTN linker
  misc_feature 316..993: note = HBsAg
  misc_feature 994..1005: note = C-tag
  misc_feature 1006..1008: note = Stop codon
  source 1..1008: mol_type = other DNA
gactccgctactcacatcaagttctccaagagagatgaggacggtaaagaattggctggtgctactatggaattgagagactcctccggtaagactatctccacttggatttccgacggtcaggttaaggacttctacttgtacccaggtaagtacactttcgttgagactgctgctccagacggttacgaagttgctactgctatcactttcactgttaacgagcagggacaggttacagttaacggtaaggctactaagggtgacgctcatattggttctggtggatctggtggttccggtccagttactaatatggaaaacatcacttccggtttcttgggtcctttgttggttttgcaggctggattcttcttgttgactagaatcttgactatcccacagtccttggactcttggtggacttccttgaacttcttgggtggttccccagtttgtttgggtcaaaactctcaatccccaacttccaaccactccccaacatcttgtccaccaatttgtcctggttacagatggatgtgtttgagaagattcatcattttcttgttcatcttgttgttgtgtttgatcttcttgttggttttgttggactaccagggtatgttgccagtttgtccattgatcccaggttccactactacaaacactggtccatgtaagacttgtactactccagctcagggtaactccatgttcccttcatgttgttgtactaagccaactgacggtaactgtacttgtatcccaattccatcctcctgggctttcgctaagtacttgtgggaatgggcttccgttagattctcctggttgtccttgttggttccattcgttcagtggttcgttggtttgtccccaactgtttggttgtccgctatttggatgatgtggtactggggtccatccttgtactctatcgtttccccattcatccctttgttgccaatcttcttctgtttgtgggtttacatcgagccagaggcttaa

SEQ ID NO: 23 - DNA, 276 bp, synthetic construct
  misc_feature 1..276: note = SpyCatcherDeltaN1
  source 1..276: mol_type = other DNA
gactccgctactcacatcaagttctccaagagagatgaggacggtaaagaattggctggtgctactatggaattgagagactcctccggtaagactatctccacttggatttccgacggtcaggttaaggacttctacttgtacccaggtaagtacactttcgttgagactgctgctccagacggttacgaagttgctactgctatcactttcactgttaacgagcagggacaggttacagttaacggtaaggctactaagggtgacgctcatatt

SEQ ID NO: 24 - DNA, 27 bp, synthetic construct
  misc_feature 1..27: note = Flexible linker from SpyCatcher-HBsAg
  source 1..27: mol_type = other DNA
ggttctggtggatctggtggttccggt

SEQ ID NO: 25 - DNA, 12 bp, synthetic construct
  misc_feature 1..12: note = PVTN linker from SpyCatcher-HbsAg
  source 1..12: mol_type = other DNA
ccagttactaat

SEQ ID NO: 26 - DNA, 678 bp, synthetic construct
  misc_feature 1..678: note = HBsAg
  source 1..678: mol_type = other DNA
atggaaaacatcacttccggtttcttgggtcctttgttggttttgcaggctggattcttcttgttgactagaatcttgactatcccacagtccttggactcttggtggacttccttgaacttcttgggtggttccccagtttgtttgggtcaaaactctcaatccccaacttccaaccactccccaacatcttgtccaccaatttgtcctggttacagatggatgtgtttgagaagattcatcattttcttgttcatcttgttgttgtgtttgatcttcttgttggttttgttggactaccagggtatgttgccagtttgtccattgatcccaggttccactactacaaacactggtccatgtaagacttgtactactccagctcagggtaactccatgttcccttcatgttgttgtactaagccaactgacggtaactgtacttgtatcccaattccatcctcctgggctttcgctaagtacttgtgggaatgggcttccgttagattctcctggttgtccttgttggttccattcgttcagtggttcgttggtttgtccccaactgtttggttgtccgctatttggatgatgtggtactggggtccatccttgtactctatcgtttccccattcatccctttgttgccaatcttcttctgtttgtgggtttacatc

SEQ ID NO: 27 - protein, 744 aa, synthetic construct
  REGION 1..744: note = gH-SpyTag-His
  SIGNAL 1..23
  DOMAIN 24..717: note = Ectodomain
  TRANSMEM 718..719: note = Transmembrane domain (truncated)
  REGION 720..725: note = misc_feature - Linker
  REGION 726..738: note = misc_feature - Spytag
  REGION 739..744: note = misc_feature - 6x His tag
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLGSGGSGAHIVMVDAYKPTKHHHHHH

SEQ ID NO: 28 - protein, 719 aa, synthetic construct
  REGION 1..719: note = gH with truncated transmembrane domain
  SIGNAL 1..23
  DOMAIN 24..717: note = Ectodomain
  TRANSMEM 718..719: note = Transmembrane domain (truncated)
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLL

SEQ ID NO: 29 - protein, 6 aa, synthetic construct
  REGION 1..6: note = Linker from gH construct
GSGGSG

SEQ ID NO: 30 - protein, 13 aa, synthetic construct
  REGION 1..13: note = Spytag
AHIVMVDAYKPTK

SEQ ID NO: 31 - protein, 278 aa, Human cytomegalovirus
  SIGNAL 1..30
  DOMAIN 31..278: note = Ectodomain
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 32 - protein, 233 aa, synthetic construct
  REGION 1..233: note = UL130 - C-tag
  SIGNAL 1..25
  DOMAIN 26..214: note = Ectodomain
  REGION 215..229: note = misc_feature - Linker
  REGION 230..233: note = misc_feature - C-tag
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIVGGGGSGGGGSGGGGSEPEA

SEQ ID NO: 33 - protein, 214 aa, synthetic construct
  REGION 1..214: note = UL130 (signal sequence and ectodomain)
  SIGNAL 1..25
  DOMAIN 26..214: note = Ectodomain
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 34 - protein, 15 aa, synthetic construct
  REGION 1..15: note = Linker from UL130 construct
GGGGSGGGGSGGGGS

SEQ ID NO: 35 - protein, 171 aa, Human cytomegalovirus
  SIGNAL 1..27
  DOMAIN 28..171: note = Ectodomain
MSPKDLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 36 - protein, 129 aa, Human cytomegalovirus
  SIGNAL 1..18
  DOMAIN 19..129: note = Ectodomain
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 37 - protein, 335 aa, synthetic construct
  REGION 1..335: note = SpyCatcher-HBsAg
  REGION 1..92: note = misc_feature - SpycatcherDeltaN1
  REGION 93..101: note = misc_feature - Flexible Linker
  REGION 102..105: note = misc_feature - PVTN linker
  REGION 106..331: note = misc_feature - HBsAg
  REGION 332..335: note = misc_feature - C-tag
DSATHIKFSKRDEDGKELAGATMELRDSSGKTISTWISDGQVKDFYLYPGKYTFVETAAPDGYEVATAITFTVNEQGQVTVNGKATKGDAHIGSGGSGGSGPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIEPEA

SEQ ID NO: 38 - protein, 92 aa, synthetic construct
  REGION 1..92: note = SpyCatcherDeltaN1
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 39 - protein, 9 aa, synthetic construct
  REGION 1..9: note = Flexible linker from SpyCatcher-HBsAg
GSGGSGGSG

SEQ ID NO: 40 - protein, 4 aa, synthetic construct
  REGION 1..4: note = PVTN linker from SpyCatcher-HBsAg
PVTN

SEQ ID NO: 41 - protein, 226 aa, synthetic construct
  REGION 1..226: note = HBsAg
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 42 - DNA, 2235 bp, synthetic construct
  misc_feature 1..2235: note = gH-(GSG)2-SpyTag-His (without introns) optimised for CHO expression
  sig_peptide 1..69
  misc_feature 70..2151: note = gH ectodomain
  misc_feature 2152..2157: note = gH truncated transmembrane domain
  misc_feature 2158..2175: note = (GSG)2 linker
  misc_feature 2176..2214: note = Spytag
  misc_feature 2215..2232: note = Histag
  misc_feature 2233..2235: note = Stop codon
  source 1..2235: mol_type = other DNA
atgaggcctggcctgccttcttatctgatcatcctggccgtgtgcctgttctcccatctgctgtcctctagatacggcgccgaggctgtgtctgagcctctggataaggcctttcatctgctgctgaacacctacggcagacctatccggttcctgcgcgagaacaccacacagtgcacctacaactccagcctgcggaactccacagtcgtgcgggaaaacgccatctccttcaactttttccagtcctacaaccagtactatgtgttccacatgcctcgctgcctgttcgctggacctctggctgagcagttcctgaaccaggtggacctgaccgagacactggaaagataccagcagcggctgaacacatatgccctggtgtctaaggacctggcctcctacagatccttcagccagcagctgaaggctcaggactctctgggagagcagcctacaacagtgcctcctcctatcgacctgtctatccctcacgtgtggatgcctccacagaccacacctcatggctggaccgagtctcataccacctctggcctgcaccggcctcacttcaaccagacctgcatcctgttcgacggccacgacctgctgttctccaccgtgacaccatgtctgcaccagggcttctacctgatcgacgagctgagatacgtgaagattaccctgacagaggacttcttcgtggtcaccgtgtccatcgacgacgacacccctatgctgctgatcttcggccatctgcctcgggtgctgttcaaggccccttaccagcgggacaacttcatcctgagacagaccgagaagcacgagctgctggtgctggtcaagaaggaccagctgaaccggcactcctacctgaaggaccctgacttcctggacgccgctctggacttcaactacctggatctgagcgccctgctgcggaacagctttcacagatacgccgtggacgtgctgaagtctggcagatgccagatgctggacagacggaccgtggaaatggccttcgcttacgccctggctctgtttgccgccgctagacaagaagaggctggcgcccaagtgtccgtgcctagagcactggatagacaagccgctctgctgcagatccaagagttcatgatcacatgcctgtctcagacccctcctcggaccacactgctgctgtatcctaccgctgtggatctggccaagagggctctgtggacccctaaccagatcaccgacatcacatccctcgtgcggctggtgtacatcctgtccaagcagaaccagcagcatctgatccctcagtgggccctgaggcagatcgctgattttgccctgaagctgcacaagacccacctggccagctttctgtctgccttcgccagacaagagctgtacctgatgggcagcctggtgcactctatgctggtgcataccaccgagcggcgcgagatcttcatcgtggaaaccggcctgtgttccctggccgagctgtctcactttacccagctgctcgctcaccctcaccacgagtacctgtccgacctgtacaccccttgctcctctagcggcagaagggaccacagcctggaaagactgacccggctgttccctgatgccaccgtgcctgctacagttcctgccgctctgtccatcctgagcaccatgcagccttccactctggaaacattccccgacctgttctgcctgcctctgggcgagtctttttctgccctgaccgtgtccgagcacgtgtcctacatcgtgaccaatcagtacctgatcaagggcatcagctaccccgtgtccacaaccgtcgttggccagagcctgatcatcacccagaccgactctcagaccaagtgcgagctgacccggaacatgcacacaacccactccatcaccgtggctctgaacatctccctggaaaactgcgccttctgccagtctgccctgctggaatacgatgacacccagggcgtgatcaacatcatgtatatgcacgactccgacgacgtgctgtttgccctggatccttacaacgaggtggtggtgtctagccccagaacacactacctgatgctgctcaagaacggcaccgtgctggaagtgaccgacgtggtggtggacgccaccgattctagattgctcggctctggtggctccggcgctcatatcgtgatggtggatgcttacaagcccaccaagcaccatcatcaccaccactaa

SEQ ID NO: 43 - DNA, 837 bp, synthetic construct
  misc_feature 1..837: note = gL (without introns) optimised for CHO expression
  sig_peptide 1..90
  misc_feature 91..834: note = gL ectodomain
  misc_feature 835..837: note = Stop codon
  source 1..837: mol_type = other DNA
atgtgcagaaggcctgactgcggcttctccttctctcccggacctgtgatcctgctgtggtgctgtctgctgctgcccatcgtttcttccgccgctgtgtctgtggctcctaccgctgctgaaaaggtgccagctgagtgtcccgagctgaccagaagatgtctgctgggcgaagtgttcgagggcgataagtacgagtcttggctgcggcctctggtcaacgtgaccggaagagatggacccctgagccagctgatccggtacagacctgtgacacctgaggccgccaattccgtgctgctggatgaggccttcctggacacactggccctgctgtacaacaaccccgatcagctgagagccctgctgaccctgctgtcctctgataccgctcctagatggatgaccgtgatgcggggctactctgagtgcggagatggaagcccagccgtgtacacctgtgtggacgatctgtgcagaggctacgacctgaccagactgtcctacggccggtccatctttaccgagcatgtgctgggctttgagctggtgcctcctagcctgttcaatgtggtggtggccatccggaatgaggccaccagaacaaatagagccgtgcggctgcctgtgtctacagctgctgctcctgagggcatcaccctgttctacggcctgtacaacgccgtgaaagagttctgcctgagacaccagctggaccctccactgctgaggcacctggataagtactacgctggcctgcctcctgagctgaagcagaccagagtgaacctgcctgctcactccagatacggccctcaggctgtggacgccagataa

SEQ ID NO: 44 - DNA, 516 bp, synthetic construct
  misc_feature 1..516: note = UL128 (without introns) optimised for CHO expression
  sig_peptide 1..81
  misc_feature 82..513: note = UL128 ectodomain
  misc_feature 514..516: note = Stop codon
  source 1..516: mol_type = other DNA
atgtcccctaaggatctgacccctttcctgaccgctctgtggctgctgctgggccattctagagtgcctagagtcagagccgaggaatgctgcgagttcatcaacgtgaaccatcctccagagcggtgctacgacttcaagatgtgcaacagattcaccgtggctctgcggtgccctgatggcgaagtgtgctactcccctgaaaagaccgccgagatcagaggcatcgtgaccaccatgacacactccctgaccagacaggtggtgcacaacaagctgaccagctgcaactacaaccctctgtacctggaagccgacggcagaatcagatgcggcaaagtgaacgacaaggcccagtacctgttgggcgctgctggctctgtgccctacagatggatcaacctggaatacgacaagatcacccggatcgtcggcctggaccagtatctggaatccgtgaagaagcacaagcggctggacgtgtgcagagccaagatgggctatatgctgcagtaa

SEQ ID NO: 45 - DNA, 702 bp, synthetic construct
  misc_feature 1..702: note = UL130-(G4S)3 - C-tag (without introns) optimised for CHO expression
  sig_peptide 1..75
  misc_feature 76..642: note = UL130 ectodomain
  misc_feature 643..687: note = (G4S)3 linker
  misc_feature 688..699: note = C-tag
  misc_feature 700..702: note = Stop codon
  source 1..702: mol_type = other DNA
atgctgagactgctgctgagacaccacttccactgcctgctgctgtgtgccgtttgggctacaccttgtctggcctctccatggtctaccctgaccgccaaccagaatccttctccaccttggtccaagctgacctactccaagcctcacgatgccgctaccttctactgcccctttctgtacccatctccacctcggagccctctgcagttctctggcttccagagagtgtccaccggacctgagtgccggaacgagacactgtacctgctgtacaaccgcgagggccagacactggtggaaagatcctctacctgggtcaagaaagtgatctggtatctgagcggccggaaccagaccatcctgcagagaatgcctcggaccgcctctaagccttctgacggcaacgtgcagatctccgtggaagatgccaagatcttcggcgcccacatggtgcccaagcagaccaaactgctgagattcgtggtcaacgacggcacccgctaccagatgtgcgtgatgaagctggaaagctgggcccacgtgttccgggattactccgtgtctttccaagtgcggctgaccttcaccgaggccaacaaccagacctacaccttctgcacccatcctaacctgatcgtcggaggcggaggatctggcggaggtggaagtggcggaggcggatctgagcccgaggcctaa

SEQ ID NO: 46 - DNA, 390 bp, synthetic construct
  misc_feature 1..390: note = UL131A (without introns) optimised for CHO expression
  sig_peptide 1..54
  misc_feature 55..387: note = UL131 ectodomain
  misc_feature 388..390: note = Stop codon
  source 1..390: mol_type = other DNA
atgagactgtgcagagtgtggctgtccgtgtgcctgtgtgctgtggttctgggccagtgccagagagagacagccgagaagaacgactactacagagtgccccactactgggacgcctgcagtagagctttgcccgatcagacccggtacaaatacgtggaacagctggtggatctgaccctgaactaccactacgacgcctctcacggcctggacaacttcgacgtgctgaagcggatcaacgtgaccgaggtgtccctgctgatctctgacttccggcggcagaatagaagaggcggcaccaacaagcggaccacctttaatgctgccggctctctggctccccacgccagatctctggaattttccgtgcggctgttcgccaactaa

SEQ ID NO: 47 - DNA, 1587 bp, synthetic construct
  misc_feature 1..1587: note = RSV-F-SpyTag-Ctag
  sig_peptide 1..75
  misc_feature 1435..1515: note = Foldon domain
  misc_feature 1516..1533: note = (GSG)2 linker
  misc_feature 1534..1572: note = Spytag
  misc_feature 1573..1584: note = Ctag
  misc_feature 1585..1587: note = Stop codon
  source 1..1587: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccggatctggcagcgccatttgcagcggcgtggccgtgtgtaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagccggaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactgcgtgaataagcaggaaggcaagagcctgtacgtgaagggcgagcccatcatcaacttctacgaccccctggtgttccccagcgacgagttcgacgctagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctgggaagcggaggctctggtgcccatatcgtgatggtggacgcctacaagcctaccaaagagcccgaggcctaa

SEQ ID NO: 48 - DNA, 1515 bp, synthetic construct
  misc_feature 1..1515: note = Sc9-10 DS-Cav1 A149C Y458C (RSV-F)
  sig_peptide 1..75
  misc_feature 1435..1515: note = Foldon domain
  source 1..1515: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccggatctggcagcgccatttgcagcggcgtggccgtgtgtaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagccggaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactgcgtgaataagcaggaaggcaagagcctgtacgtgaagggcgagcccatcatcaacttctacgaccccctggtgttccccagcgacgagttcgacgctagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctg

SEQ ID NO: 49 - DNA, 1440 bp, synthetic construct
  misc_feature 1..1440: note = Sc-9-10 DS-Cav1 A149C Y458C (RSV-F) without the signal peptide
  misc_feature 1360..1440: note = Foldon domain
  source 1..1440: mol_type = other DNA
cagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccggatctggcagcgccatttgcagcggcgtggccgtgtgtaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagccggaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactgcgtgaataagcaggaaggcaagagcctgtacgtgaagggcgagcccatcatcaacttctacgaccccctggtgttccccagcgacgagttcgacgctagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctg

SEQ ID NO: 50 - protein, 528 aa, synthetic construct
  REGION 1..528: note = RSV-F-SpyTag-Ctag
  SIGNAL 1..25
  DOMAIN 479..505: note = Foldon domain
  REGION 506..511: note = MISC_FEATURE - (GSG)2 linker
  REGION 512..524: note = MISC_FEATURE - Spytag
  REGION 525..527: note = MISC_FEATURE - Ctag
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFLGSGGSGAHIVMVDAYKPTKEPEA

SEQ ID NO: 51 - protein, 1010 aa, synthetic construct
  REGION 1..1010: note = Sc-9-10 DS-Cav1 A149C Y458C (RSV-F)
  SIGNAL 1..25
  DOMAIN 479..505: note = Foldon domain
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLMELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 52 - protein, 480 aa, synthetic construct
  REGION 1..480: note = sc9-10 DS-Cav1 A149C Y458C (RSV-F) without the signal peptide
  DOMAIN 454..480: note = Foldon domain
QNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 53 - DNA, 1704 bp, synthetic construct
  misc_feature 1..1704: note = RSV-F DS-Cav1-SpyTag-Ctag
  sig_peptide 1..75
  misc_feature 1552..1632: note = Foldon domain
  misc_feature 1633..1650: note = (GSG)2 linker
  misc_feature 1651..1689: note = Spytag
  misc_feature 1690..1701: note = Ctag
  misc_feature 1702..1704: note = Stop codon
  source 1..1704: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccaacaacagagccagaagagagctgccccggttcatgaactacaccctgaacaacgccaagaaaaccaacgtgaccctgagcaagaagagaaagagaagattcctgggcttcctgctgggcgtgggcagcgccattgccagcggcgtggccgtgtgtaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactacgtgaataagcaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctgggaagcggaggctctggtgcccatatcgtgatggtggacgcctacaagcctaccaaagagcccgaggcctaa

SEQ ID NO: 54 - DNA, 1632 bp, synthetic construct
  misc_feature 1..1632: note = RSV-F DS-Cav1
  sig_peptide 1..75
  misc_feature 1552..1632: note = Foldon domain
  source 1..1632: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccaacaacagagccagaagagagctgccccggttcatgaactacaccctgaacaacgccaagaaaaccaacgtgaccctgagcaagaagagaaagagaagattcctgggcttcctgctgggcgtgggcagcgccattgccagcggcgtggccgtgtgtaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactacgtgaataagcaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctg

SEQ ID NO: 55 - DNA, 1557 bp, synthetic construct
  misc_feature 1..1557: note = RSV-F DS-Cav1 without the signal peptide
  misc_feature 1477..1557: note = Foldon domain
  source 1..1557: mol_type = other DNA
cagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccaacaacagagccagaagagagctgccccggttcatgaactacaccctgaacaacgccaagaaaaccaacgtgaccctgagcaagaagagaaagagaagattcctgggcttcctgctgggcgtgggcagcgccattgccagcggcgtggccgtgtgtaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactacgtgaataagcaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctg

SEQ ID NO: 56 - protein, 567 aa, synthetic construct
  REGION 1..567: note = RSV-F DS-Cav1-SpyTag-Ctag
  SIGNAL 1..25
  DOMAIN 518..544: note = Foldon domain
  REGION 545..550: note = MISC_FEATURE - Linker
  REGION 551..563: note = MISC_FEATURE - Spytag
  REGION 564..567: note = MISC_FEATURE - Ctag
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGSGGSGAHIVMVDAYKPTKEPEA

SEQ ID NO: 57 - protein, 544 aa, synthetic construct
  REGION 1..544: note = RSV-F DS-Cav1
  SIGNAL 1..25
  DOMAIN 518..544: note = Foldon domain
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 58 - protein, 519 aa, synthetic construct
  REGION 1..519: note = RSV-F DS-Cav1 without the signal peptide
  DOMAIN 493..519: note = Foldon domain
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 59 - DNA, 2088 bp, synthetic construct
  misc_feature 1..2088: note = gH without the signal peptide
  misc_feature 1077: note = C>A mutation at position 1077
  misc_feature 1..2082: note = Ectodomain
  misc_feature 2083..2088: note = Transmembrane domain (truncated)
  source 1..2088: mol_type = other DNA
cgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacatgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctc

SEQ ID NO: 60 - protein, 696 aa, synthetic construct
  REGION 1..696: note = gH without the signal peptide
  DOMAIN 1..694: note = Ectodomain
  TRANSMEM 694..696: note = Transmembrane domain (truncated)
RYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLL

(19) United States
(12) Patent Application Publication
(10) Pub. No.: US 2026/0007740 A1
(43) Pub. Date: Jan. 8, 2026

(54) VACCINE COMPOSITION

(71) Applicant: SpyBiotech Limited, Oxford (GB)

(72) Inventors: Sumi BISWAS, Oxford (GB); Jing JIN, Oxford (GB); Rebecca Alice DABBS, Oxford (GB); Genevieve Marie Catherine LABBÉ, Oxford (GB)

(73) Assignee: SpyBiotech Limited, Oxford (GB)

(21) Appl. No.: 19/323,644

(22) Filed: Sep. 9, 2025

Related U.S. Application Data
(63) Continuation of application No. 17/052,707, filed on Nov. 3, 2020, now Pat. No. 12,433,944.

(30) Foreign Application Priority Data
May 4, 2018 (GB) 1807376.7
May 4, 2018 (GB) 1807378.3

(51) Int. Cl. A61K 39/245; A61K 39/00; A61K 39/12; A61P 31/14; A61P 31/22

(52) U.S. Cl. CPC A61K 39/245; A61K 39/12; A61P 31/14; A61P 31/22; A61K 2039/5258; A61K 2039/545; C12N 2710/16123; C12N 2710/16134; C12N 2760/18523; C12N 2760/18534

(57) ABSTRACT

The present invention relates to vaccine compositions, most notably vaccine compositions wherein the antigenic component is large, for example over 50 kDa, or multimeric, i.e. comprised of subunits. Such antigenic components are of particular interest, because they may represent antigenic components from pathogens that currently it is not possible to vaccinate against. The invention relates to a composition comprising a particle displaying an antigenic component, wherein said composition comprises an antigenic component comprising a first peptide tag, and a moiety comprising a second peptide tag, wherein the antigenic component and the moiety are linked via an isopeptide bond between said first and second peptide tags, and wherein the antigenic component is over 50 kDa, or alternatively is multimeric.

### CROSS-REFERENCE TO RELATED APPLICATIONS

This application is a continuation of U.S. application Ser. No. 17/052,707 filed on Nov. 3, 2020, which is the U.S. national stage entry of International Application No. PCT/GB2019/051245 filed on May 3, 2019, incorporated by reference herein, PCT/GB2019/051245 claiming the benefit of priority to GB Application No. 1807376.7 filed on May 4, 2018, and GB Application No. 1807378.3 filed on May 4, 2018.

### SEQUENCE LISTING

This application contains a sequence listing, submitted electronically in XML format under the file name “00012-0050_Sequence Listing.xml”, which is incorporated by reference herein in its entirety. The XML copy of the sequence listing was created on Sep. 9, 2025, and is 148,959 bytes in size.

### BACKGROUND TO THE INVENTION

Vaccines are a safe and effective way to combat and eradicate infectious diseases. Vaccine development has been very successful, but there is a list of remaining disease challenges against which no vaccine currently exists, including many important pathogens representing daunting immunological obstacles. It is generally considered that an effective vaccine must traffic to lymph nodes, persist for a sufficient time to generate an immune response.

Vaccine development has moved away from using attenuated or dead pathogens to using smaller antigenic components of those pathogens, in an aim to generate the required protective immune response whilst avoiding the risks inherent by using such attenuated strains. Efforts have focussed on attempting to express immunogenic portions of components of a pathogen (such as those components which are required for the pathogen to infect a cell) which, for simplicity, have been limited to short/small peptides and proteins because of technical problems with the expression of large or multicomponent antigens. However, a potential problem with using very short or small peptides is the risk of antigenically variable pathogens, which escape the immune response induced by vaccination through changes in that particular part of the antigen.

There would be immunological advantages associated with expression of large/multicomponent antigens, including the ability to allow the generation of antibodies against multiple neutralising epitopes of the one pathogen. However, expression of one or more large antigens which may form a complex, in such a way that the relevant antigenic epitopes are maintained and presented for generation of an effective antibody response remains a huge challenge. There therefore remains a need for improved methods for expressing large antigens and/or multicomponent antigens in such a way that they can raise a clinically significant immune response.

Previous recombinant vaccines designed to invoke an immune response against multiple antigenic components either rely on each component being expressed and packaged separately into distinct particles, for example in the case of the anti-HPV vaccines Cervarix and Gardasil, where recombinant major capsid L1 proteins of particular HPV strains are separately expressed and assembled as virus-like particles (VLP), following which the different types of VLP are combined into the vaccine formulation. Alternatively, multiple short epitopes are selected and combined into a single recombinant vaccine (e.g. the Multimeric-001 influenza vaccine), but by their very nature these epitopes are short linear peptides, chosen in order to avoid manufacturing complexities involved with three-dimensional structures or refolding, and therefore do not attempt to represent the native pathogen as presented to the immune system in active infection.

As an example, the β-herpes human Cytomegalovirus (HCMV, also known as human herpesvirus-5 (HHV-5)) is a leading viral cause of neonatal developmental disabilities. This ubiquitous virus has infected over 60% of the general population, with initial infection usually being only minor or asymptomatic. After infection, the virus remains latent in the body but can cause serious disease in the immunocompromised (i.e. HIV patients, transplant patients and those undergoing chemotherapy) or elderly. HCMV is the leading infectious cause of birth defects in developed countries. Up to 4/200 babies are born with HCMV due to congenital infection, and up to 10% of these will suffer long term consequences. HCMV infection has also been implicated in high blood pressure and atherosclerosis in adults (Cheng et al. (May 2009). Früh K, ed. “Cytomegalovirus infection causes an increase of arterial blood pressure”. PLoS Pathog. 5 5):e1000427). HCMV is therefore a public health priority. Despite intensive efforts, however, a successful HCMV vaccine has not been developed to date.

Respiratory syncytial virus (RSV) is another ubiquitous virus that causes very little ill health in healthy adults and older children that it infects. However, it is the second largest cause of death in infants under the age of one worldwide, second only to Malaria. The virus is responsible for an estimated 160,000 deaths per year worldwide. This virus causes serious respiratory infections, and complications include pneumonia and bronchiolitis. High risk groups include infants under the age of one and immunocompromised patients, the elderly, and those with heart and lung conditions. Again, no currently licensed vaccine for RSV exists despite many years of active research and development.

For diseases such as those caused by RSV and HCMV, where there are no currently available vaccines, generally current approaches to vaccine production haven't shown the desired efficacy, indicating that there is a large unmet need in providing an alternative type of vaccine in order to deal with diseases with such catastrophic outcomes.

Recently several genetically-encoded systems for enabling spontaneous or assisted amide bond formation have been described. For example, SpyTag is a peptide which has been engineered such that a spontaneous and irreversible isopeptide bond to its protein partner SpyCatcher is formed when the two components are mixed. The position of the SpyTag and SpyCatcher components within protein chains can be designed to be at various locations and are reactive under a wide range of pH, buffer and temperature conditions. The SpyTag/SpyCatcher pair and variants and derivatives thereof have been used in vaccine development but only for the presentation of simple antigens to date. Other genetically encoded systems for enabling spontaneous amide bond formation include SnoopTag/SnoopTagJr and SnoopCatcher; RrgATag/RrgATag2/DogTag and RrgACatcher, IsopepTag/IsopepTag-N and Pilin-C or Pilin-N, PsCsTag and PsCsCatcher; and SnoopTagJr and DogTag (mediated by SnoopLigase), and variants of all these systems.

The present inventors have proven that use of large/multicomponent antigens in vaccine compositions is possible using genetically-encoded systems for enabling amide bond formation, which may improve the response to the large/multicomponent antigen. This is a surprising result.

### SUMMARY OF THE INVENTION

In a first aspect of the invention there is provided a composition comprising a particle displaying a protein component, wherein said composition comprises:
i) a protein component comprising a first peptide tag, and
ii) a moiety comprising a second peptide tag,
wherein the protein component and the moiety are linked via an isopeptide bond between said first and second peptide tags, and wherein the protein component is over 50 kDa.

In another aspect of the invention there is provided a composition comprising a particle displaying a protein component, wherein said composition comprises:
i) a protein component comprising a first peptide tag, and
ii) a moiety comprising a second peptide tag,
wherein the protein component and the moiety are linked via an isopeptide bond between said first and second peptide tags, and wherein the protein component is multimeric.

The protein component may have any function e.g. it may be an enzyme or have enzymatic properties. The protein component may be a full-length protein, or it may be a part, segment, domain or truncation of a full-length protein. The protein component may be an antigen or an immunogen. The protein component may also be called the antigenic component.

In another aspect of the invention there is provided a composition comprising a particle displaying an antigenic component, wherein said composition comprises:
i) an antigenic component comprising a first peptide tag, and
ii) a moiety comprising a second peptide tag,
wherein the antigenic component and the moiety are linked via an isopeptide bond between said first and second peptide tags, and wherein the antigenic component is over approximately 50 kDa.

In some embodiments of any aspect of the invention the protein component or the antigenic component may be over 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa or more, such as over 200 kDa, over 300 kDa or over 400 kDa.

A multimer may comprise any number of subunits, which may or may not be covalently linked in the protein or antigenic component. The multimer may comprise 2-20 subunits, alternatively, 3, 4, 5, 6, 7, 8, 9, 10, 11, 12, 13, 14, 15 or more subunits. Alternatively, the multimer may be a dimer, trimer, tetramer, pentamer, hexamer, septamer, octamer, nonamer or decamer. The multimer may be from any appropriate pathogen, but is preferably a viral multimer.

Non-limiting examples of large protein components, i.e. those over 50 kDa or as described above, include the pentameric complex (PC) and gB glycoprotein from Human cytomegalovirus (HCMV), the G and F glycoproteins from RSV, the haemagglutinin (HA) and neuraminidase (NA) antigens from influenza A virus, Plasmodium falciparum Pfs230, P. falciparum CSP, Human HER2 receptor, PCSK9, VAR2CSA, P. falciparum RIPR, Varicella zoster virus (VZV) glycoprotein E, Rabies virus glycoprotein and the Epstein-Barr virus (EBV) gH/gL complex.

In some embodiments of any aspect of the invention the protein component or the antigenic component may be a monomer or a multimer, for example a dimer, trimer, tetramer or pentamer. In some embodiments of any aspect of the invention the protein component or the antigenic component may be a protein or peptide complex.

Non-limiting examples of multimeric antigenic components include the pentameric complex (PC) and gB trimer from Human cytomegalovirus (HCMV), the G and F glycoproteins from RSV, the haemagglutinin (HA) and neuraminidase (NA) antigens from influenza A virus, some of which are described herein. Other examples include components of disease agents such as viruses, bacteria, fungal pathogens, parasites or other disease vectors. Suitable multimeric antigenic components include, for example those derived from viruses such influenza (such as Influenza hemagglutinin (HA) (e.g. Flu trimer)), Respiratory syncytial virus (RSV) etc.

The protein component may be attached to the first peptide tag by genetic fusion, and expressed recombinantly in an appropriate cell. For components that include post-translational modifications such as glycosylation it may be preferable to express the recombinant protein in a eukaryotic or mammalian cell line.

In one embodiment, the “moiety” is a component onto which a protein component or an antigenic component may be displayed e.g. made available to the immune system. In one embodiment the moiety multimerises to form said particle. Suitably, such a moiety may be a virus, a bacteria, a multimerisation scaffold for vaccination or a protein component which multimerises to form a VLP (virus-like particle). Suitably, the moiety may be a component of a bacteriophage, tobacco mosaic virus particle, adeno-associated virus like particles (AAVLP), E. coli etc. In one embodiment, the moiety is itself a component of the virus, bacteria etc. such that the multimerisation (e.g. self-assembly) of the moiety forms the particle for displaying the protein component or the antigenic component. In one embodiment the moiety may be a viral structural protein, for example a viral envelope or capsid protein or surface antigen. Examples of structural proteins include matrix M1 protein and viral envelope M2 protein from the influenza virus, HBsAg from Hepatitis B virus, E. coli bacteriophage AP205 viral coat protein (CP3), hemagglutinin-neuraminidase from a variety of viruses including Mumps and the like. Suitable viral structural proteins will be known to those skilled in the art. In other embodiments, the moiety may be a protein or peptide, such as a multimerisation domain such as IMX313, which forms nanoparticles, or a computationally derived particle such as MI3. In further embodiments the moiety may be a synthetic nanoparticle or a synthetic VLP, such as a gold, lipopeptide or poly(lactic-co-glycolic acid) (PLGA) nanoparticle. Other suitable moieties may include liposomes or outermembrane vesicles. Suitably a moiety comprising a second peptide tag is a moiety to which a second peptide tag is attached.

The use of a structural surface antigen from a virus may be preferred. The second peptide tag is therefore attached to the structural surface antigen, permitting the formation of a virus-like particle (VLP), to which the second peptide tag is attached, or to which is displaying the second peptide tag. VLPs are non-infectious self-assembling nanoparticles and their repetitive molecularly-defined architecture is attractive for engineering multivalency, notably for vaccination. VLPs have been produced from components of a wide variety of virus families including the Hepatitis B virus (including Hepatitis B small surface antigen (HBsAg)), Parvoviridae (e.g. adeno-associated virus), Retroviridae (e.g. HIV), Flaviviridae (e.g. Hepatitis C virus) and bacteriophages (e.g. Qβ, AP205). Any of these may be suitable for use as the moiety in the present invention.

The second peptide tag may be attached to the moiety through genetic fusion. This genetic fusion may be at any appropriate point in the sequence, not simply limited to the termini. Those skilled in the art will appreciate that the fusion protein may be expressed recombinantly in an appropriate cell.

The second peptide tag may alternatively be displayed or attached to the moiety by means of chemical conjugation. This would require, for example, the presence of a reactive amine group in order to allow the conjugation to take place.

Accordingly, in one embodiment, the moiety is a surface antigen of the hepatitis B virus (HBsAg). Suitably HBsAg has the amino acid sequence set out in SEQ ID NO:41, as described herein (or functional equivalents thereof).

In one embodiment of any aspect of the invention the protein component or antigenic component is an immunogenic component of an HCMV pentamer. Suitably, an antigenic or immunogenic component is one which is capable of generating an immune response such as an antibody response against that component upon introduction into a subject such as a patient. Accordingly, an “immunogenic component of an HCMV pentamer”, for example, is a component which is capable of generating an anti-HCMV antibody response in a subject. Suitably an immunogenic component comprises one or more of (at least one of) the HCMV pentamer subunit components selected from gH, gL, pUL128, pUL130 and pUL131 (also known as pUL131A). In some embodiments, the immunogenic component comprises one or more of those “pUL” or “UL” components. In other embodiments, the immunogenic component comprises one or more of those gH or gL components. In one embodiment the immunogenic component comprises a combination of one or more “UL” components with one or more components selected from the gH or gL components. In another embodiment of the invention, the immunogenic component of an HCMV pentamer is the HCMV pentamer comprising all of the gH/gL/pUL128/pUL130/pUL131 subunits. Suitably the gH/gL/pUL128/pUL130/pUL131 subunits have amino acid sequences corresponding to those derived from any known HCMV strain (including both laboratory strains and/or clinical isolates), including Towne (GI:239909366), AD169 (GI:219879600), Toledo (GI:290564358) and Merlin (GI:155573956), or functional equivalents thereof. By functional equivalents is meant amino acid sequences that share some homology and differ only in some amino acids but retain the functional property of being able to form an antigenic subunit or pentamer that provides protective antibodies. Suitable variants of the components gH/gL/pUL128/pUL130/pUL131A are described, for example, in WO2014/005959 (see pages 4 to 10), hereby incorporated by reference. Advantageously, using HCMV pentamer subunits in a vaccine approach can provide immunogenic protection against infection from a wide range of HCMV viral strains, due to the high degree of homology between strains at the level of pentamer amino acid sequence.

In some embodiments, an antigenic component may correspond to a component of a disease agent or vector or a portion thereof. An antigenic component may, for example, lack a transmembrane domain for ease of manufacture. Suitably, in the HCMV pentamer, for example, the immunogenic component of an HCMV pentamer comprises a gH subunit with a truncated transmembrane domain (having been truncated by deletion of one or more amino acids from this region) such that the subunit is secreted into the cell supernatant, during protein production in host cells, for ease of purification.

In one embodiment, the gH/gL/pUL128/pUL130/pUL131A subunits have the amino acid sequences set out in SEQ ID NOs:28, 31, 35, 33, 36, respectively (or functional equivalents thereof) (with or without the signal peptide indicated). By functional equivalents is meant amino acid sequences that share some homology and differ only in some amino acids but retain the functional property e.g. of being able to form an antigenic subunit or pentamer that provides protective antibodies. In some embodiments, a functional equivalent may share 70%, 80%, 90% homology, or more, with the relevant amino acid sequence. In another embodiment, the gH/gL/pUL128/pUL130/pUL131A subunits are encoded by nucleic acid sequences such as those set out in SEQ ID NOs:13, 16, 20, 18, 21, or codon optimised versions thereof (with or without the encoding sequence for the signal peptide). In some embodiments any one of the gH/gL/pUL128/pUL130/pUL131A subunits may have a signal peptide, for example that signal peptide present on the native protein for that strain, a functional equivalent of the signal peptide, or a signal peptide derived from a different strain of HCMV. In some embodiments any one of the gH/gL/pUL128/pUL130/pUL131A subunits may have a signal peptide derived from a heterologous protein. The choice of signal peptide may be determined in order to target the expressed protein to a particular cellular (or extracellular) location, or to confer other functionality. Following expression of the subunit(s), the signal peptide may be enzymatically cleaved (e.g. by a signal peptidase), either by native cellular machinery in the expression system used, or in vitro. In some embodiments, any one of the gH/gL/pUL128/pUL130/pUL131A subunits may be expressed without a signal peptide. In some embodiments, the native sequences, including introns, may be used where these may result in higher expression levels. Suitably, the native nucleic acid sequence for UL128 includes 2 introns. In another embodiment, the nucleic acid sequence for UL131A includes one intron. In some embodiments, the introns may be removed. In some embodiments, the native sequences may be codon-optimised for the relevant expression system.

In one embodiment of any aspect of the invention the protein component or antigenic component is an immunogenic component of a RSV virus, such as the attachment glycoprotein (G protein) or fusion glycoprotein (F protein), both of which control the initial phase of infection. G is a highly glycosylated 90 kDa type II integral membrane protein, and can mediate viral attachment to the host cell membrane either through interaction with heparan sulphate on proteoglycans, and is a good candidate for a protein component.

The F protein is an integral membrane protein composed of three Fo monomers that are processed during assembly into F1 and F2 subunits, which are covalently linked by two disulphide bonds. The F protein is highly conserved amongst RSV isolates from both A and B subgroups and the amino acid sequences show 90% or above identity. F is a 574 amino acid class I fusion protein consisting of a 50 kilodalton (kDa) carboxy-terminal F1 fragment and a 20 kDa amino-terminal F2 fragment; making it a trimer of heterodimers. It is distinguished by two furin cleavage sites that liberate a 27 amino acid glycopeptide and expose the hydrophobic fusion peptide at the F1 amino terminus. There are two N-linked glycosylation sites in F2 and only one in F1. After removal of the 25 amino acid signal peptide and the 27 amino acid glycopeptide between F2 and F1, the remaining ectodomain of F consists of 472 amino acids. Only 25 amino acids in the Fectodomain differ between subtypes A and B.

In order to develop an antigenic composition from RSV-F protein, some studies have focussed upon making variants of the pre-fusion protein, which is a trimer. Variants have been produced by genetically fusing the two subunits of mature pre-F into a single chain. DS-Cav1 variants with F2 fused genetically to F1 and both fusion peptide and pep27 region deletions have been made. Differences in the linker between F2 and F1 subunits appeared to affect immunogenicity, and therefore variants may use a selection of different linkers. The native RSV-F protein sequence may be found at Accession number P03420.1.

Several versions of pre-fusion F proteins have been researched and developed, and consequently published. These pre-fusion trimers may all be suitable for use in the present invention. The DS-Cav1-stabilized fusion glycoprotein is derived from the native protein. EP2222710, incorporated here by reference, also discloses recombinant RSV antigen comprising a soluble F protein polypeptide comprising an F2 domain and an F1 domain of an RSV-F protein polypeptide and a trimerisation domain. In Nat. Commun. 2015; 6:8143, Krarup et al, a highly stable pre-fusion RSV-F protein is described, again incorporated by reference.

WO2014/160463, herein incorporated by reference, describes isolated recombinant RSV-F proteins that are stabilised in a pre-fusion conformation, as well as nucleic acid molecules encoding the recombinant RSV-F proteins.

WO2017/172890, herein incorporated by reference, describes substitution-modified pre-fusion RSV-F proteins, and nucleic acids coding therefor. Further description is given in Nat Struct Mol Biol. 2016 September; 23(9):811-820, Iterative structure-based improvement of a respiratory syncytial virus fusion glycoprotein vaccine, M. Gordon Joyce, Baoshan Zhang, Li Ou, Man Chen, Gwo-Yu Chuang, Aliaksandr Druz, Wing-Pui Kong, Yen-Ting Lai, Emily J. Rundlet, Yaroslav Tsybovsky, Yongping Yang, Ivelin S. Georgiev, Miklos Guttman, Christopher R. Lees, Marie Pancera, Mallika Sastry, Cinque Soto, Guillaume B. E. Stewart-Jones, Paul V. Thomas, Joseph G. Van Galen, Ulrich Baxa, Kelly K. Lee, John R. Mascola, Barney S. Graham, and Peter D. Kwong, also incorporated herein by reference.

Exemplary nucleic acid sequences encoding recombinant F₂-F₁ ectodomain protomers linked to a T4 Fibritin trimerization domain are available as Accession Numbers: LP884611.1, LP884610.1, LP884609.1 and LP884608.1.

In some embodiments, a protein or antigenic component may correspond to a component of a disease agent or vector or a portion thereof. An antigenic component may, for example, lack a transmembrane domain for ease of manufacture. Suitably, in the RSV-F protein or a pre-fusion conformation thereof, for example, the immunogenic component of an F protein comprises a F₂-F₁ subunit with a truncated transmembrane domain (having been truncated by deletion of one or more amino acids from this region) such that the subunit is secreted into the cell supernatant, during protein production in host cells, for ease of purification. Therefore, the RSV-F protein lacks a functional TM domain. Alternatively, the genetic fusion with the first peptide tag may indeed prevent the F protein from residing in the membrane despite the presence of a functional transmembrane domain.

In one embodiment, the pre-fusion stabilised subunits have the amino acid sequences set out in SEQ ID NO:50-58, respectively (or functional equivalents thereof). By functional equivalents is meant amino acid sequences that share some homology and differ only in some amino acids but retain the functional property e.g. of being able to form an antigenic subunit that provides protective antibodies. In some embodiments, a functional equivalent may share 70%, 80%, 90% homology, or more, with the relevant amino acid sequence. In one embodiment, the pre-fusion stabilised RSV-F trimer may not include a heterologous trimerisation domain.

The protein component comprises a first peptide tag. This first peptide tag may be attached to the protein component by expressing a recombinant fusion protein. Those skilled in the art will be aware of techniques for the genetic fusion of peptide sequences in order to express the recombinant protein in suitable cell systems. For moieties that include post-translational modifications such as glycosylation it is preferable to express the recombinant protein in a eukaryotic or mammalian cell line.

Advantageously using a first peptide tag and a second peptide tag which form an isopeptide bond, such as the SpyTag-SpyCatcher system as described herein, allows “decoration” of the large and/or multimeric antigen, such as the HCMV pentamer, or immunogenic component thereof, onto the moiety which displays said large antigen, such as a VLP, in the correct formation and orientation such that the antigen is presented to the immune system in such a way as to be able to generate anti-antigen (e.g. anti-HCMV) antibodies which can provide a protective/neutralising/immunogenic effect. Traditional approaches of vaccination that use a soluble antigen (even a large antigen such as a multimer/pentamer) may be less effective in producing a protective/neutralising/immunogenic effect. Advantageously, display of an antigen (e.g. a multimeric antigen) on a particle, such as a VLP or nanoparticle, results in the presentation of a geometric repetitive array of identical antigens that, in contrast to soluble antigens, are capable of robustly triggering an immune response. The larger size of VLPs or other suitable particles compared to ‘free’ antigens may also have a greater immunogenic effect. In addition, the orientation of display of a multimeric antigen, e.g. the HCMV pentamer, may be important to immunogenicity. The use of paired tags, such as the SpyTag-SpyCatcher system as described herein, to attach a multimeric antigen onto a particle permits the antigen to be attached to the particle in a particular advantageous orientation. For example in the case of HCMV, the gH/gL subunits may be less likely to have neutralising epitopes than the “UL” subunits. Thus, advantageously, the present invention permits the orientation of display of the HCMV pentamer on a particle to be determined by suitable positioning of the first peptide tag, such that, for example, the “UL” subunits are displayed towards the outside of the particle and therefore are more easily available to the immune system of an individual. Alternatively/additionally, the positioning of the first peptide tag on the antigen may be determined in order to produce a similar orientation of the antigen to that on the native virus, thereby presenting to the immune system a particle displaying an antigen in an orientation more likely to induce an immune response to an invading live virus.

In contrast, traditional approaches for presenting a protein onto a VLP may involve chemical linkage which has the disadvantages that such a chemical reaction may be more random, such that the correct (e.g. immunologically preferred) orientation of the antigen could not be obtained with certainty and may only represent a small proportion of the linkage reactions obtained. Moreover, the processes involved in a chemical conjugation may make it unlikely that the 3-D structure required for suitable antigen presentation could be maintained. Some disadvantages of traditional approaches are described, for example, in Brune et al. 2016; Scientific Reports, 6:19234, DOI:10.1038/srep19234, Brune et al. Bioconjugate Chemistry, 2017, 28, 1544-1551, and Leneghan et al (2017) Scientific reports, 7:3811.

Similarly, genetic fusion of an antigen to a viral coat protein has proved challenging and time-consuming because of problems with misfolding and in determining expression conditions optimal for both of the two components. Moreover a genetic fusion would not be appropriate for expression of a large antigen or a multi-component antigen as effective expression in correct conformation would be too difficult to achieve.

In order to present the protein or antigenic component in such a way that it is immunogenic, the position of the first peptide tag needs to be carefully designed such that the native protein conformation is maintained, and optionally any post-translational modifications are retained, if appropriate. For some antigens, the retention of glycosylation does not affect the way the epitopes are presented, but for others either maintaining or removing them improves efficacy. For protein components that are transmembrane proteins, the transmembrane section of the protein component provides a good target for positioning the first peptide tag, since this sequence is not involved with the conformation of the protein component that is antigenic, and provides a role that will no longer be required in a vaccine, for example. If the protein component does not include a transmembrane protein, fusing the first peptide tag to a C- or N-terminus of the component or a subunit thereof (in the case of a multimer) may prove helpful, but the first peptide tag can also be included in any part of the sequence. Alternatively, it may be possible to locate the first peptide tag in a loop on the protein or antigenic component.

In order to present an immunogenic component, such as an immunogenic component of an HCMV pentamer, the position of the first peptide tag needs to be carefully designed such that the native protein conformation is maintained. For HCMV, in one embodiment, attachment is via the gH subunit, suitably via the C-terminus of the gH subunit, or transmembrane domain (or portion thereof) of the gH subunit. In addition to maintaining the conformation of the pentamer (or component of the pentamer), this rational design also presents the target region of the pentamer towards the outside of the particle as discussed above. As used herein, the target region is the part of the protein known to raise antibodies with a neutralising effect, and may also be referred to as the immunogenic portion.

In order to present an immunogenic component, such as an immunogenic component of an RSV pre-fusion F protein, the position of the first peptide tag needs to be carefully designed such that the native protein conformation is maintained. For RSV-F pre-fusion protein, in one embodiment, attachment suitably via the C-terminus of the F pre-fusion, via the 3′ end of the nucleic acid encoding the same. In addition to maintaining the conformation of the pre-fusion F protein (or component thereof), this rational design also presents the most neutralising epitopes of the pre-fusion F protein towards the outside of the particle as discussed above. The same considerations will apply for any other variation of the F protein. Inclusion of the first peptide tag at the C-terminus has been demonstrated to work by the present inventors, leaving an immunogenic protein component to fold correctly.

In one embodiment the first and second peptide tags are part of a peptide tag/binding partner pair capable of forming an isopeptide bond. This isopeptide bond may be spontaneous, i.e. without assistance, or require assistance, i.e. from a ligase or other helper. Suitably, the first and second peptide tag are a SpyTag/SpyCatcher pair. Suitably, the first and second peptide tag are selected from the list comprising SpyTag/SpyCatcher, SnoopTag/SnoopTagJr and SnoopCatcher; RrgATag/RrgATag2/DogTag and RrgACatcher, IsopepTag/IsopepTag-N and Pilin-C or Pilin-N, PsCsTag and PsCsCatcher; and SnoopTagJr and DogTag (mediated by SnoopLigase), and variants, derivatives and modifications of all these systems.

Suitably, the first peptide tag is the peptide tag from a peptide tag/binding partner pair, such as SpyTag, and the second peptide tag is the binding partner, such as SpyCatcher. In another embodiment, the first peptide tag is the binding partner, such as SpyCatcher, and the second peptide tag is the peptide tag component from the peptide tag/binding partner pair, such as SpyTag. Suitably, the first peptide tag is the peptide tag from a peptide tag/binding partner pair, such as SnoopTag, and the second peptide tag is the binding partner, such as SnoopCatcher. In another embodiment, the first peptide tag is the binding partner, such as SnoopCatcher, and the second peptide tag is the peptide tag component from the peptide tag/binding partner pair, such as SnoopTag. Thus, it can be seen that the first peptide tag can be either the “tag” or “catcher”; with the second peptide tag being the partner for this pair, the “catcher” or the “tag”, respectively. Suitable peptide tag/binding partner pairs are described in detail in WO2011/09877, WO2016/193746, WO2018/18951 and WO2018/197854, herein incorporated by reference.

In one embodiment, the protein or antigenic component is attached to any one of SpyTag, SnoopTag, RrgATag, RrgATag2, DogTag, IsopepTag, IsopepTag-N, PsCsTag and SnoopTagJr as a first peptide tag.

The first peptide tag may be attached via a linker, if required, which may be rigid or flexible. Those skilled in the art will appreciate which linker would be appropriate.

In another embodiment, the moiety is attached to any one of SpyCatcher, SnoopCatcher, RrgACatcher, Pilin-C, Pilin-N, PsCsCatcher and DogTag (mediated by SnoopLigase) as a second peptide tag.

The moiety may be any suitable moiety, as discussed previously, including synthetic multimerisation platforms.

The second peptide tag may be attached to any suitable position in the moiety, which does not affect its ability to fold and form an appropriate conformation. Genetic fusion may be preferred. It may be preferable to include the second peptide tag at the C- or N-terminus of the moiety but the second peptide tag can also be included in any part of the sequence. Alternatively, it may be possible to locate the second peptide tag in a loop on the moiety. For example, genetically fused SpyCatcher to the N-terminus of the viral coat protein (CP3) of the RNA bacteriophage AP205 is described in Brune et al, Scientific Reports volume 6, Article number: 19234 (2016). Alternative fusions using self-assembling synthetic proteins as multimerisation platforms are discussed in Bruun et al, ACS Nano, 2018, 12 (9), pp 8855-8866. The second peptide tag may alternatively be attached via chemical conjugation.

The second peptide tag may be attached via a linker, if required, which may be rigid or flexible. Those skilled in the art will appreciate which linker would be appropriate.

In one embodiment, the antigenic component, such as the HCMV pentamer or immunogenic component thereof, is attached to SpyTag. A suitable SpyTag has the amino acid sequence set out in SEQ ID NO:30.

The SpyTag may be attached via a linker. Suitable linkers include the linker having the amino acid sequence set out in SEQ ID NO:29.

In another embodiment, the moiety is attached to a SpyCatcher binding partner (second peptide tag). The moiety may suitably be HBsAg. A suitable SpyCatcher has the amino acid sequence set out in SEQ ID NO:38. In one embodiment, SpyCatcher is attached via a linker. The linker may be a rigid linker or a flexible linker, suitably wherein the linker has the amino acid sequence set out in SEQ ID NO:39.

In another embodiment, the protein composition or antigenic composition in accordance with any aspect or embodiment of the invention further comprises another, preferably different, protein comprising a first peptide tag.

In another embodiment, the composition in accordance with any aspect or embodiment of the invention further comprises another, preferably different, antigen comprising a first peptide tag, such as another HCMV antigen. Suitably the other HCMV antigen is glycoprotein B. Suitably glycoprotein B sequences are described, for example, in WO2014/005959, see SEQ ID NOs:21, 22, 23 or 36. In one embodiment, the composition comprises particles (e.g. VLPs) displaying both the HCMV pentamer and the other HCMV antigen.

In one embodiment, the composition is an immunogenic composition or vaccine composition. Preferably said immunogenic or vaccine composition is one which is capable of inducing an immune response, such as an antibody response, upon administration to an individual. Suitably the immune response may be a protective immune response. A suitable immunogenic composition may further comprise additional components including adjuvants, immunostimulants and/or pharmaceutically acceptable excipients.

Suitable adjuvants, for example, may be based on aluminium, peptides, squalene, liposomes, oil-in-water emulsions and saponin, and may include Alhydrogel®, MF59, AS01, MatrixM, muramyl dipepide and Quil A. Water-in-oil adjuvants are also suitable. Squalene-Oil-in-water emulsions, such as Addavax™, are suitable.

Accordingly, in another aspect or embodiment of the invention there is provided an immunogenic or vaccine composition comprising a composition in accordance with the invention. Suitably, a vaccine composition comprises a vaccine dose which is an amount of composition in accordance with the invention which provides an immunogenic, preferably immunoprotective effect from an infective agent/vector, such as a neutralising effect from HCMV infection. Suitably, a vaccine composition comprises a vaccine dose which is an amount of composition in accordance with the invention which provides a neutralising effect from an infective agent/vector, such as a neutralising effect from RSV infection. Antibodies, preferably neutralising antibodies generated to an immunogenic composition may be detected and measured by methods familiar to those skilled in the art, including standardised ELISA assays or microneutralisation assays, as described herein, for example.

In another aspect there is provided a VLP comprising:
(i) a moiety comprising a first peptide tag
(ii) a protein comprising a second peptide tag
wherein said first peptide tag and said second peptide tag form an isopeptide bond. In some embodiments, the moiety is HBsAg. However, any suitable moiety may be used, as described previously.

Suitably, the first peptide tag is the peptide tag from a peptide tag/binding partner pair, such as SpyTag, and the second peptide tag is the binding partner, such as SpyCatcher. In another embodiment, the first peptide tag is the binding partner, such as SpyCatcher, and the second peptide tag is the peptide tag from the peptide tag/binding partner pair, such as SpyTag. Other suitable peptide tag/binding partner pairs are described herein and will be known to those skilled in the art. Suitably, the first and second peptide tag are selected from the list comprising SpyTag/SpyCatcher, SnoopTag/SnoopTagJr and SnoopCatcher; RrgATag/RrgATag2/DogTag and RrgACatcher, IsopepTag/IsopepTag-N and Pilin-C or Pilin-N, PsCsTag and PsCsCatcher; and SnoopTagJr and DogTag (mediated by SnoopLigase), and variants, derivatives and modifications of all these systems.

Suitably the protein comprising the second peptide tag is a protein or peptide complex which is greater than 50 kDa. The protein comprising the second peptide tag may be a protein or peptide complex which is greater than 50 kDa, 60 kDa, 70 kDa, 80 kDa, 90 kDa, 100 kDa, 110 kDa, 120 kDa, 130 kDa, 140 kDa, 150 kDa or 160 kDa, 170 kDa, 180 kDa, 190 kDa or more, such as over 200 kDa, over 300 kDa or over 400 kDa.

In one embodiment, the protein comprising the second peptide tag is a multimeric protein. In one embodiment, the protein comprising the second peptide tag is an antigen, preferably a multimeric antigen. Suitably, the multimeric antigen may be HCMV pentamer as described herein. Suitably, the protein may be an RSV-F protein or derivative thereof (such as the pre-fusion F protein). In one embodiment, the protein comprising a second peptide tag is an immunogenic component of HCMV pentamer. The HCMV pentamer (gH/gL/pUL128/pUL130/pUL131) as described herein and including suitable linkers and tags has a molecular weight of over 160 kDa. Other suitable large or multimeric proteins or antigens include antigens from other infectious agents including viruses such as influenza virus, RSV and so forth.

Advantageously, using HBsAg as a carrier (VLP) in this way would also be likely to generate an anti-HepB boost, alternatively described as an anti-Hepatitis B virus (HBV) response.

In another aspect there is provided a VLP comprising:
(i) a protein comprising a first peptide tag
(ii) a moiety comprising a second peptide tag
wherein said first peptide tag and said second peptide tag form an isopeptide bond. In some embodiments, the moiety is HBsAg. However, any suitable moiety may be used, as described previously.

Suitably, the first peptide tag is the peptide tag from a peptide tag/binding partner pair, such as SpyTag, and the second peptide tag is the binding partner, such as a SpyCatcher. In another embodiment, the first peptide tag is the binding partner, such as a SpyCatcher, and the second peptide tag is the peptide tag from the peptide tag/binding partner pair, such as SpyTag. Other suitable peptide tag/binding partner pairs are described herein and will be known to those skilled in the art. Suitably, the first and second peptide tag are selected from the list comprising SpyTag/SpyCatcher, SnoopTag/SnoopTagJr and SnoopCatcher; RrgATag/RrgATag2/DogTag and RrgACatcher, IsopepTag/IsopepTag-N and Pilin-C or Pilin-N, PsCsTag and PsCsCatcher; and SnoopTagJr and DogTag (mediated by SnoopLigase), and variants, derivatives and modifications of all these systems.

Suitably the protein comprising the first peptide tag is a protein or peptide complex which is greater than 50 kDa. The protein comprising the first peptide tag may be a protein or peptide complex which is greater than 50 kDa, 60 kDa, 70 kDa, 80 kDa, 90 kDa, 100 kDa, 110 kDa, 120 kDa, 130 kDa, 140 kDa, 150 kDa or 160 kDa or more, notably 200 kDa, 300 kDa or even 400 kDa or more. In one embodiment, the protein comprising the first peptide tag is a multimeric protein. In one embodiment, the protein comprising the second peptide tag is an antigen, preferably a multimeric antigen. Suitably, the multimeric antigen may be HCMV pentamer as described herein. Suitably, the protein may be an RSV-F protein or derivative thereof (such as the pre-fusion F protein). In one embodiment, the protein comprising a first peptide tag is an immunogenic component of HCMV pentamer. The HCMV pentamer (gH/gL/pUL128/pUL130/pUL131A) as described herein and including suitable linkers and tags has a molecular weight of over 160 kDa. Other suitable large or multimeric proteins or antigens include antigens from other infectious agents including viruses such as influenza virus, RSV and so forth.

Advantageously, using HBsAg as a carrier (VLP) in this way would also be likely to generate an anti-HBV boost.

In another aspect of the invention, there is provided an HCMV pentamer linked to a SpyTag, as described herein.

In accordance with another aspect of the invention there is provided a method of producing a composition or VLP in accordance with the invention, said method comprising: introducing a first nucleic acid which encodes a first genetic fusion of a first protein to a first peptide tag into a first host cell;
incubating said first host cell under conditions for expressing said first genetic fusion; optionally purifying the expressed components;
introducing a second nucleic acid which encodes a second genetic fusion of a second protein to a second peptide tag into a second host cell;
incubating said second host cell under conditions for expressing said second genetic fusion; optionally purifying the expressed components;
incubating the expressed components under conditions for formation of an isopeptide bond between the first peptide tag and the second peptide tag; optionally purifying the resultant composition.

Suitably, the expressed components are incubated together in order for the isopeptide bond to form. The formation of the isopeptide bond may require co-incubation with a ligase or similar.

Suitably, the method of producing a composition or VLP in accordance with the invention may be for producing a composition comprising an antigenic component displayed on a VLP.

In some embodiments, where the “immunogenic component of the HCMV pentamer” comprises the entire HCMV pentamer, recombinant production of the components of the HCMV pentamer requires each subunit to be expressed in the right stoichiometry for the pentamer to be formed, as well as to fold correctly for assembly. In these embodiments, complexes of just parts of the required pentamer (e.g. gH/gL dimers and tetramers, or tetramers lacking any one of the five subunits) need to be excluded from the final product. Advantageously, the present invention overcomes the problems that would otherwise be associated by expressing all of the vaccine components in one system (i.e. HBsAg and 5 subunits of HCMV pentamer) by providing a simple approach of making the components separately and then conjugating them. Accordingly, in one embodiment, a purification tag is incorporated onto UL130 (Hofmann et al, DOI 10.1002/bit 25670). Similar principles would be applicable to other immunogenic components.

In some embodiments, where the “immunogenic component of the RSV-F protein” comprises the entire F protein or a derivative thereof, recombinant production of the components of the F protein or derivative thereof requires it to fold correctly for assembly, with derivatives including the pre-fusion F protein trimer.

Suitably, the method is for producing a composition comprising an HCMV pentamer displayed on an HBsAg VLP. Suitably, the method is for producing a composition comprising an RSV-F pre-fusion F protein trimer displayed on an HBsAg VLP.

In another aspect of the invention there is provided a vaccine for use in the prophylaxis and/or treatment of a disease. Suitably, said vaccine comprises a composition or VLP in accordance with any aspect or embodiment of the invention. In one embodiment, the disease is HCMV infection. In another aspect there is provided a prophylactic method of treatment for HCMV. Suitably, the vaccine is for use in humans. Suitably the vaccine is for use in adult humans, for example women of reproductive age or pregnant women. In another aspect, the invention provides a method of inducing an immunogenic response, for example a protective immune response, for HCMV in an individual wherein the method comprises administering a composition in accordance with any aspect or embodiment of the invention.

In another aspect of the invention there is provided a composition in accordance with any aspect of the invention for use as a medicament.

In a further aspect of the invention there is provided a composition in accordance with any aspect of the invention for use as a vaccine, preferably a vaccine for use in prophylaxis and/or treatment of HCMV infection. A composition for use as a medicament or a vaccine in accordance with the invention may be administered to human adults, for example women of reproductive age or pregnant women.

In another aspect, the invention provides nucleic acid molecules for use in a method in accordance with the invention. In one embodiment, a nucleic acid molecule in accordance with the invention comprises a nucleic acid sequence encoding an amino acid sequence as set out in any of SEQ ID NOs:27 to 41. In one embodiment, a nucleic acid molecule in accordance with the invention comprises a nucleic acid sequence as set out in any of SEQ ID NOs:12 to 26 or 42 to 46.

In another aspect, the invention provides a plurality of nucleic acid molecules comprising those nucleic acid molecules encoding an amino acid sequence as set out in SEQ ID NOs:27 to 41. In one embodiment, the nucleic acid molecules of the invention include those having a sequence as set out in any of SEQ ID NOS:12 to 26 or 42 to 46.

In another aspect, the invention provides nucleic acid molecules for use in a method in accordance with the invention. In one embodiment, a nucleic acid molecule in accordance with the invention comprises a nucleic acid sequence encoding an amino acid sequence as set out in any of SEQ ID NOs:50 to 58. In one embodiment, a nucleic acid molecule in accordance with the invention comprises a nucleic acid sequence as set out in any of SEQ ID NOs:47 to 55.

In another aspect, the invention provides a plurality of nucleic acid molecules comprising those nucleic acid molecules encoding an amino acid sequence as set out in SEQ ID NOs:50 to 58. In one embodiment, the nucleic acid molecules of the invention include those having a sequence as set out in any of SEQ ID NOS:47 to 55.

In another aspect, the invention provides a vector comprising a nucleic acid molecule or a plurality of nucleic acid molecules in accordance with the invention. Suitably a vector is an expression vector for expressing the amino acid sequence of any component of a composition in accordance with the invention.

In another aspect, the invention provides host cells for expressing the components of a composition in accordance with the invention. Suitable host cells may be those for transient or stable expression of those components. Methods and host cells for expressing CMV proteins are described, for example, in WO2014/005959 and WO2016/067239, both incorporated by reference. In some embodiments, the components may be glycosylated.

In another aspect of the invention, there is provided a kit comprising a composition in accordance with the invention for use in a prime-boost vaccination regime. Suitably said kit may comprise a prime composition comprising a first immunogenic composition in accordance with the invention and a boost composition comprising a second immunogenic composition in accordance with the invention. Alternatively, the kit may be provided to provide a single or multiple dose vaccination regime, i.e., 1, 2, 3, 4, 5, 6, 7, 8, 9, or 10 doses. Accordingly, in another aspect the invention provides a dosage regime comprising doses applied at approximately 3 week intervals.

### FIGURES

FIG. 1. SDS-PAGE and Western blot analysis of purified pentamer-SpyTag under non-reducing and reducing conditions. Lane 1: ColorPlus Prestained Broad Range Protein Ladder, sizes indicated in kDa; Lane 2: non-reduced sample; Lane 3: reduced sample. A) SDS-PAGE and Coomassie staining analysis, with the position of the HCMV pentamer components indicated, non-reduced to the left and reduced to the right of the gel. B) Western blot analysis using anti-HCMV pentamer antibody.

FIG. 2. SDS-PAGE and Western blot analysis of purified SpyCatcher-HBsAg under non-reducing (NR) and reducing conditions (R). A) SDS-PAGE and Coomassie staining analysis. B)

Western blot analysis using anti-HBsAg monoclonal antibody.

FIG. 3: HPLC analysis using a s200increase 3.2/300 column. A) 10 μl of purified HCMV pentamer-SpyTag was loaded and eluted as a single peak. B) 10 μl of purified SpyCatcher-HBsAg was loaded and eluted as a single main peak at the void volume of the column.

FIG. 4: SDS-PAGE and Western-blot analysis of conjugated pentamer-SpyTag and SpyCatcher-HBsAg under reducing conditions. 1: ColorPlus Prestained Broad Range Protein Ladder, sizes indicated in kDa; 2: conjugation; 3: pentamer-SpyTag; 4: SpyCatcher-HBsAg. A) SDS-PAGE and Coomassie staining analysis. B) Western blot using anti-HBsAg monoclonal antibody. C) Western blot using anti-pentamer polyclonal antibody.

FIG. 5: HPLC analysis using a s200 increase 3.2/300 column. 30 μl of conjugated pentamer-SpyTag--SpyCatcher-HBsAg was loaded and eluted as a main peak at the void volume of the column.

FIG. 6: Immunogenicity of HCMV pentamer-HBsAg vaccine versus pentamer protein vaccine, adjuvanted with Addavax, after a single immunisation. BALB/c mice were immunised with 1 μg or 0.1 μg of HCMV pentamer-SpyTag, either as soluble protein or as a pentamer-HBsAg VLP. Titres were measured by standardised ELISA from mice sera. Lines represent the means, error bars represent the standard deviation (n=10). Mice immunised with HCMV pentamer-HBsAg VLP show substantially stronger serum IgG antibody responses compared to mice immunised with HCMV Pentamer protein alone, even when the pentamer-equivalent VLP dose is 10× lower.

FIG. 7: Neutralising activity in the sera of mice immunised with HCMV pentamer-HBsAg vaccine compared to pentamer protein vaccine. Vaccines were adjuvanted with Addavax, and responses shown after one (post-prime) or two immunisations (post-boost). NT₅₀ was measured on ARPE-19 cells infected with AD169^{wt131} strain (displaying a functional pentamer). Neutralising titres for Cytogam and a commercially available neutralising anti-gH mAb (HCMV16 (51C1) from Bio-Rad Antibodies) in the same assay are indicated.

FIG. 8: Immunogenicity of HCMV pentamer-HBsAg vaccine versus pentamer protein vaccine, unadjuvanted, after one or two immunisations. BALB/c mice were immunised with 1 μg or 0.1 μg of HCMV pentamer-SpyTag conjugated to SpyCatcher-HBsAg (Pentamer-HBsAg), or with 1 μg of pentamer-SpyTag protein. Titres were measured by standardised ELISA from mice sera. Lines represent the means, error bars represent the standard deviation (n=10). Mice immunised with HCMV pentamer-HBsAg VLP show substantially stronger serum IgG antibody responses compared to mice immunised with HCMV Pentamer protein alone, even when the pentamer-equivalent VLP dose is 10× lower.

FIG. 9: Neutralising activity in the sera of mice immunised with HCMV pentamer-HBsAg vaccine compared to pentamer protein vaccine. Vaccines were unadjuvanted, and the response shown after one (post-prime) or two immunisations (post-boost). NT50 was measured on ARPE-19 cells infected with AD169^{wt131} strain (displaying a functional pentamer). Neutralising titres for Cytogam and a commercially available neutralising anti-gH mAb (HCMV16 (51C1) from Bio-Rad Antibodies) in the same assay are indicated.

FIG. 10. SDS-PAGE and Western blot analysis of purified RSV-F-SpyTag under non-reducing and reducing conditions. A) SDS-PAGE and Coomassie staining analysis, Lane 1: ColorPlus Prestained Broad Range Protein Ladder; Lane 2: non-reduced sample; Lane 3: reduced sample. B) Western blot analysis using anti-RSV-F monoclonal antibody, Lane 1: ColorPlus Prestained Broad Range Protein Ladder; Lane 2: non-reduced sample; Lane 3: reduced sample.

FIG. 11. SDS-PAGE and Western-blot analysis of RSV-F-SpyTag conjugated with SpyCatcher-HBsAg under reducing conditions. 1: ColorPlus Prestained Broad Range Protein Ladder, 2: RSV-F-SpyTag--SpyCatcher-HBsAg conjugate, 3: RSV-F-SpyTag, 4: SpyCatcher-HBsAg. A) SDS-PAGE and Coomassie staining analysis. B) Western blot using anti-HBsAg monoclonal antibody. C) Western blot using anti-RSV-F monoclonal antibody.

FIG. 12. Immunogenicity of conjugated RSV-F-SpyTag--SpyCatcher-HBsAg (‘Sc9-10-HBsAg’) versus unconjugated RSV-F-SpyTag (‘Sc9-10’). BALB/c mice were immunised with 1 μg of RSV-F-SpyTag conjugated to SpyCatcher-HBsAg (RSV-F VLP) or 1 μg of RSV-F-SpyTag protein, either unadjuvanted or with Addavax™ (n=8). RSV-F antigen is sc9-10 DS-Cav1 A149C Y458C-SpyTag.

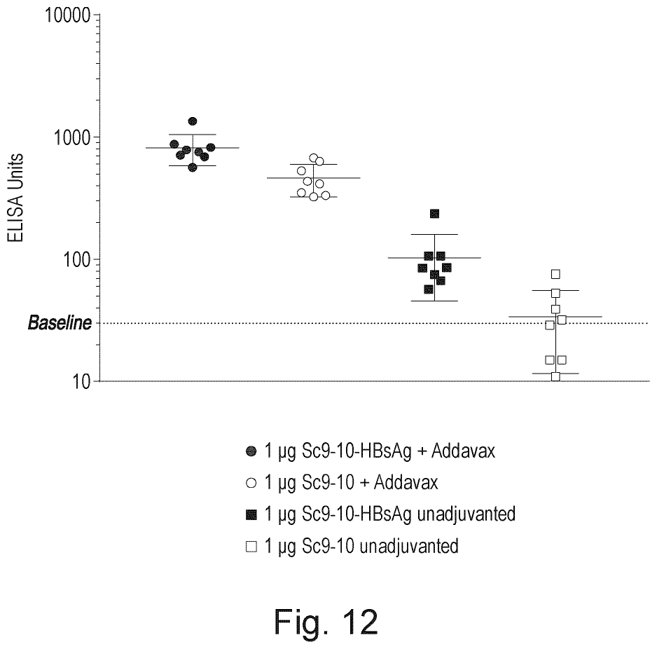

### DETAILED DESCRIPTION OF THE INVENTION

### Virus-Like Particles

Traditionally, vaccine approaches used attenuated or dead whole pathogens although this has been replaced by using recombinant subunit vaccines which include a protein from the appropriate pathogen. More recently, approaches using Virus-like particles (VLPs) have been developed. VLPs are particles which resemble viruses in their size (approx. 20-200 nm), their shape and their repetitive protein arrangement but lack any genetic material from a pathogen.

Because of their size, VLPs are more likely to drain to lymph nodes, making them ideal for uptake and presentation by antigen-presenting cells. In addition, their repetitive structure facilitates complement fixation and B cell receptor cross-linking (Kushnir et al. Vaccine 2012; Vol 31(1):58-83). However, their mechanism of action is not restricted to theory.

### HCMV

Human Cytomegalovirus (HCMV, also known as human herpesvirus-5 (HHV-5)) is a virus that most adults have been exposed to, with initial infection usually being only minor or asymptomatic. After infection, the virus remains latent in the body but can cause serious disease in the immunocompromised or elderly. HCMV is also the leading infectious cause of birth defects in developed countries. Up to 4/200 babies are born with HCMV due to congenital infection, and up to 10% of these will suffer long term consequences. HCMV infection has also been implicated in high blood pressure and atherosclerosis in adults (Cheng et al. (May 2009). Früh K, ed. “Cytomegalovirus infection causes an increase of arterial blood pressure”. PLoS Pathog. 5 (5):e1000427).

The pentameric complex of HCMV comprising the viral protein gH/gL/pUL128/pUL130/pUL131A has been identified as a potentially useful vaccine target for HCMV based on the observation that antibodies to this complex can neutralise the entry of virus into epithelial cells as well as reduce the risk of the transmission of HCMV perinatally. Despite intensive efforts, however, a successful HCMV vaccine has not been developed to date.

### HCMV Pentamer

HCMV strains, including clinical isolates and laboratory strains, differ in the sequence of their genomes. HCMV strains include Merlin (GI:155573956), Towne (GI239909366) and AD169 (GI:219879600), Toledo (GI290564358) and TB40/E. HCMV contains multiple membrane proteins and protein complexes. The pentameric protein gH/gL/pUL128/pUL130/pUL131A is important for HCMV infection of epithelial and endothelial cells, thought to be through endocytic pathways. Other combinations of the components of this complex have been shown to be important for infection of e.g. fibroblast cells. “pUL” subunits/components are also referred to as “UL”; “pUL131” is also referred to as “pUL131A” and “pUL131a”, or “UL131A”.

Various HCMV strains have been deposited with the ATCC, and can be found as: Merlin (VR-1590), Towne (VR-977) and AD169 (VR-538). Genomic sequences may be reference via accession numbers: Merlin (AY446894.2), Towne (GO121041.1), AD169 (FJ527563.1), Toledo (GU37742.2) and TB40/E (KF297339.1).

### RSV

Respiratory syncytial virus is a leading cause of serious respiratory disease in young children throughout the world. An estimated 3.4 million children younger than 5 years of age are hospitalized each year with severe RSV lower respiratory tract infection, with the highest incidence in children younger than 6 months of age. Most deaths occur in infants under the age of 1 and in developing countries. At present, options for prevention and control are limited.

### RSV-F Pre-Fusion Trimer

The F glycoprotein is a type I viral fusion protein. It is thought that the RSV F precursor (F0) is cleaved by a furin-like protease at two sites, which generates three fragments. The shorter, N-terminal fragment (F2) is covalently attached to the larger, C-terminal fragment (F1) by two disulphide bonds. The intervening fragment of 27 amino acids dissociates after cleavage and is not found in the mature protein.

Numerous stabilised pre-fusion F trimers are available, as discussed previously. In the examples filed here, exemplary sequences encoding for these pre-fusion trimers are found as SEQ ID Nos:48, 48, 54 and 55. Sequences including a fusion with a SpyTag are included as SEQ ID NOs:47 and 53. The amino acid sequences are shown as SEQ ID Nos:51, 52, 57 and 58 for the pre-fusion trimer, and SEQ ID Nos:50 and 56 with a SpyTag. Other exemplary sequences are referred to herein.

### Peptide Tag/Binding Partner Pairs

Proteins that are capable of spontaneous isopeptide bond formation (so-called “isopeptide proteins”) have been advantageously used to develop peptide tag/polypeptide binding partner pairs (i.e. two-part linkers) which covalently bind to each other and provide irreversible interactions (see e.g. WO2011/098772 and WO 2016/193746 both herein incorporated by reference, together with WO2018/189517 and WO2018/197854 both incorporated herein by reference). In this respect, proteins which are capable of spontaneous isopeptide bond formation may be expressed as separate fragments, to give a peptide tag and a polypeptide binding partner for the peptide tag, where the two fragments are capable of covalently reconstituting by isopeptide bond formation, thereby linking molecules or components fused to the peptide tag and its polypeptide binding partner. The isopeptide bond formed by the peptide tag and its polypeptide binding partner is stable under conditions where non-covalent interactions would rapidly dissociate, e.g. over long periods of time (e.g. weeks), at high temperature (to at least 95° C.), at high force, or with harsh chemical treatment (e.g. pH 2-11, organic solvent, detergents or denaturants).

Isopeptide bonds are amide bonds formed between carboxyl/carboxamide and amino groups, where at least one of the carboxyl or amino groups is outside of the protein main-chain (the backbone of the protein). Such bonds are chemically irreversible under typical biological conditions and they are resistant to most proteases. As isopeptide bonds are covalent in nature, they result in some of the strongest measured protein-protein interactions.

In brief, a two-part linker, i.e. a peptide tag and its polypeptide binding partner (a so-called peptide tag/binding partner pair) may be derived from a protein capable of spontaneously forming an isopeptide bond (an isopeptide protein), wherein the domains of the protein are expressed separately to produce a peptide “tag” that comprises one of the residues involved in the isopeptide bond (e.g. an aspartate or asparagine, or a lysine) and a peptide or polypeptide binding partner (or “catcher”) that comprises the other residue involved in the isopeptide bond (e.g. a lysine, or an aspartate or asparagine) and at least one other residue required to form the isopeptide bond (e.g. a glutamate). Mixing the peptide tag and binding partner results in the spontaneous formation of an isopeptide bond between the tag and binding partner. Thus, by separately incorporating the peptide tag and binding partner into different molecules or components, e.g. proteins, it is possible to covalently link said molecules or components together via an isopeptide bond formed between the peptide tag and binding partner, i.e. to form a linker between the molecules or components incorporating the peptide tag and binding partner.

The spontaneous formation of the isopeptide bond may be in isolation, and not require the addition of any other entity. For some peptide tag and tag partner pairs, the presence of a helper entity, such as a ligase, may be required in order to generate the isopeptide bond.

A peptide tag/binding partner pair (two-part linker), termed SpyTag/SpyCatcher, has been derived from the CnaB2 domain of the Streptococcus pyogenes FbaB protein (Zakeri et al., 2012, Proc Natl Acad Sci U S A 109, E690-697) and used in diverse applications including vaccine development (Brune et al., 2016, Scientific reports 6, 19234; Thrane et al., 2016, Journal of Nanobiotechnology 14, 30).

Suitably, the first and second peptide tags form the peptide tag/binding pair termed

SpyTag/SpyCatcher. Suitably, the SpyCatcher component is DeltaN1 (ΔN1) SpyCatcher (as described in Li, L., Fierer, J. O., Rapoport, T. A. & Howarth, M. Structural analysis and optimization of the covalent association between SpyCatcher and a peptide Tag. J. Mol. Biol. 426, 309-317 (2014)) which has a 23 amino acid truncation at the N-terminal compared to “SpyCatcher” (SEQ ID No. 38).

In other embodiments, the first and second peptide tags form a peptide tag/binding pair which is a mutated version of SpyTag/SpyCatcher displaying an increased rate of reaction for isopeptide bond formation such as, for example, those described in co-pending application, GB1706430.4. In some embodiments, these mutated forms may be useful for the attachment of large proteins (e.g. >50 kDa or >100 kDa, such as the >160 kDa HCMV pentameric protein as described herein) and/or where slow reactions or steric hindrance may be an issue.

In other embodiments, the isopeptide proteins may include SnoopTag/SnoopCatcher, described, for example in WO 2016/193746.

In some embodiments, one or both of the isopeptide proteins may have N- or C-terminal truncations, whilst still retaining the reactivity of the isopeptide bond.

Exemplary first and second peptide tag pairs (peptide tag/binding partner pairs; reactive pairs) are described in the following table:

Reactive pairs

| | | |
|---|---|---|
| (a) | SpyTag | SpyCatcher |
| | SpyTag002 | |
| | SpyTag002 RG T3H | |
| (b) | SpyTag | SpyCatcher002 |
| | SpyTag002 | |
| | SpyTag002 RG T3H | |
| (c) | SpyTag | SpyCatcher002 D5A |
| | SpyTag002 | A92P Q100D |
| | SpyTag002 RG T3H | |
| (d) | SnoopTag | SnoopCatcher |
| | SnoopTagJr | |
| (e) | RrgATag | RrgACatcher |
| | RrgATag2 | |
| | DogTag | |
| (f) | Isopeptag | Pilin-C |
| (g) | Isopeptag-N | Pilin-N |
| (h) | PsCsTag | PsCsCatcher |
| (i) | SnoopTagJr | DogTag [mediated by |
| | | SnoopLigase] |

described, for example, in WO2011/098772, WO2016/193746, GB1706430.4 GB 1705750.6 or Li, L., et al., J. Mol. Biol. 426, 309-317 (2014).

Variants, derivatives and modifications of the binding pairs may be made by any suitable means. Variants, derivatives and functionally operative modifications may involve amino acid additions, substitutions, alterations or deletions that retain the same function in relation to the ability to form an isopeptide bond with the relevant binding partner.

For some of the binding pairs, mediation by a third entity such as an enzyme is required. For example, SnoopLigase may be used to meditate the bond formation between SnoopTagJr and DogTag. Thus, the pairing may require the assistance of an enzyme such as a ligase.

### HBsAg

By “HBsAg” is meant a surface antigen from Hepatitis B Virus (HBsAg), or portion thereof. In one embodiment, HBsAg may refer to the N-terminus of HBsAg, such as the HBsAg sequence as set out in SEQ ID NO:41, comprising 226 amino acids of the S protein of Hepatitis B virus (adw serotype). Suitably, the HBsAg includes a four amino acid sequence, Pro Val Thr

Asn, representing the four carboxy terminal residues of the hepatitis B virus (adw serotype) preS2 protein, as described in Valenzuela et al., (1979) ‘Nucleotide sequence of the gene coding for the major protein of hepatitis B virus surface antigen’ Nature 280:815-819. VLPs formed from HBsAg have been approved for clinical use against Hepatitis B (Kushnir et al. Vaccine 2012; Vol 31(1):58-83) including Recombivax HB (https://vaccines.procon.org/sourcefiles/recombivax_package_insert.pdf), and Energix B (https://au.gsk.com/media/217195/engerix-b_pi_006_approved.pdf). HBsAg has also been used as the basis for the pre-erythrocytic malaria vaccine RTS,S which has completed phase III clinical trials and is the most advanced malaria vaccine to date (http://www.malariavaccine.org/sites/www.malariavaccine.org/files/content/page/files/RTSS%20FAQs_FINAL.pdf; Kaslow and Biernaux, Vaccine 2015, Vol. 33(52):7425-7432).

### Linker Details

The distance between proteins (e.g. VLP and decorating antigen), can have an effect on the availability of antigenic epitopes in the protein, stability of the protein/s and may also have an effect on conjugation efficiency due to the accessibility of either of the isopeptide bond partners (e.g. SpyTag/SpyCatcher). Therefore a linker may be chosen with suitable properties in order to optimise availability, stability and/or accessibility. Linkers may be broadly subdivided into flexible and rigid subtypes.

### Flexible Linkers

Flexible linkers may be used when the linked domains require movement. They usually consist of small non-polar (e.g.: Gly) or polar (eg: Ser, Thr) amino acids, where the small size provides flexibility (Chen et al., 2013 Adv Drug Deliv Rev. October 15; 65(10):1357-1369). The addition of Ser or Thr can help maintain stability in solution, and adjusting the length can impact the proper folding of proteins (Chen et al., 2013). Any suitable flexible linker may be used, with the nature and length appropriate to the entities concerned. Suitably, a flexible linker may include combinations between 2 and 70 amino acids of such type.

### EXAMPLES

| Sequence name | Sequences | SEQ ID No |
|---|---|---|
| Flexible linker 1 | GSG | n/a |
| Flexible linker 2 - | GSGGSG | SEQ ID NO: 1 |
| (GSG)₂ | | |
| Flexible linker 3 - | GSGGSGGSG | SEQ ID NO: 2 |
| (GSG)₃ | | |
| Flexible linker 4 - | GGGGS | SEQ ID NO: 3 |
| (G45)₁ | | |
| Flexible linker 5 - | GGGGSGGGGSGGGGS | SEQ ID NO: 4 |
| (G45)₃ | | |
| Flexible linker 6 - | GGGGSGGGGSGGGGS | SEQ ID NO: 5 |
| (G4S)₄ | GGGGS | |
| Flexible linker 7 | GSAGSAAGSGEF | SEQ ID NO: 6 |
| Flexible linker 8 | KESGSVSSEQLAQFR | SEQ ID NO: 7 |
| | SLD | |
| Flexible linker 9 | EGKSSGSGSESKST | SEQ ID NO: 8 |

### Rigid Linkers

In some cases rigid linkers may be preferred, as they can assist with providing protein separation. Rigid linkers have a secondary structure. One of the most common rigid linkers is (EAAAK)ₙ (where n is the number of repeats) which adopts an α-helical structure (Arai et al., (2001) Protein Eng. August; 14(8):529-32). Other rigid linkers may include proline rich sequences such as (XP)ₙ, where X is any amino acid but preferentially Ala (A), Lys (K) or Glu (E), where the proline provides conformational constraint (Chen at al., 2013).

Other suitable linkers are described, for example, by Klein et al. (2014) Protein Eng Des Sel. October; 27(10):325-330. Any suitable rigid linker may be used, with the nature and length appropriate to the entities concerned. Suitably, a rigid linker may include combinations between 2 and 70 amino acids of such type.

### Examples

| Sequence name | Sequences | SEQ ID No |
|---|---|---|
| Rigid linker 1 | EAAAK | SEQ ID NO: 9 |
| Rigid linker 2 - | EAAAKEAAAKEAAAK | SEQ ID NO: 10 |
| (EAAAK)₃ | | |
| Rigid linker 3 - | APAPAPAPAPAPAP | SEQ ID NO: 11 |
| (AP)₇ | | |

### Host Cells and Expression Vectors

Suitably host cells for expression of nucleic acids to produce proteins and compositions in accordance with the invention will be known by those skilled in the art.

In one embodiment, the host cells will be suitable for transient expression. In another embodiment, host cells will be those cells which are capable of forming stable cell lines. Suitably, the coding sequences encoding the antigenic component, such as the HCMV pentamer and the RSV-F protein, including those comprising the isopeptide bond forming peptide tag will be integrated into one host cell. In one embodiment, each of the nucleic acid sequences encoding a subunit of the multimer such as a pentamer will be encompassed in a different plasmid/vector such that transfection of a host cell with, for example, all 5 plasmids/vectors will result in the pentamer being produced by the host cell, when it is cultured in suitable conditions. In other embodiments, a plasmid/vector may comprise a combination of one or more coding sequence such that at least 1, 2, 3, 4 or 5 plasmids may be introduced. Alternatively, an entire fusion peptide coding sequence may be provided in one vector, such that the entire protein component and first peptide tag are encoded on the same vector.

In one embodiment, these vectors are used for stable integration of the coding sequences into the genome of the host cells. Suitable host cells for stable expression include mammalian cells, such as HEK cells (Human embryonic kidney 293 cells) or rodent cells including CHO (Chinese Hamster Ovary) cells. Suitable mammalian cells and vectors for expression of the protein components of the composition in accordance with the invention will be known by those skilled in the art and are described, for example in WO2016/067239, at pages 15-16 and Hofmann et al., (2015) Biotech and Bioeng, 112(12):2505-2515. Exemplary stable construct sequences for expression of components in accordance with the invention may be found in Example 3 below.

### Affinity Purification

In some embodiments, those expression constructs for use in expressing components of the composition in accordance with the invention may include “tag” sequence or sequences which facilitate purification such as affinity purification. Any suitable tag, such as an affinity tag may be included in order to separate the protein component and first peptide tag from the system in which it is produced. Those skilled in the art of recombinant protein production are aware of systems such as His-tags and Strep-tags which may be included for purification purposes. Such tags dramatically aid in protein purification and rarely adversely affect biological or biochemical activity, and are therefore desirable. Suitable tag sequences include C-tag, histidine tags (His-tag), streptavidin tags (Strep-tags), maltose-binding protein (MBP), Glutathione-S-transferase (GST) and FLAG tags.

Both the protein component and/or the moiety may include an affinity purification tag. For ease of use, these are generally fused genetically at the C- or N-terminal end of the protein.

Therefore, in some embodiments, for example, the gH, gL, pUL128, pUL130, pUL131A (or a fragment thereof) subunits of HCMV, the RSV pre-fusion F protein, or the HBsAg peptides/proteins may comprise additional amino acid residues, at the N- or C-terminus, which facilitate purification. Such additional amino acid residues may comprise a tag such as a His-tag or C-tag, for example. In some embodiments, C-tag may provide a cleaner purification. Other suitable tag sequences include maltose-binding protein (MBP), Strep-tag, Glutathione-S-transferase (GST) and FLAG tag. In some embodiments, a tag may be linked to the amino acid sequence in such a way that it may be cleaved after purification e.g. by using a cleavable linker, for example. In other embodiments, non-affinity purification methods may be used.

In other embodiments, the RSV pre-fusion F protein may comprise additional amino acid residues, at the C- or N-terminus, which facilitate purification. Exemplified herein, the RSV pre-fusion F protein has a C-Tag for affinity purification.

### Conjugation of First and Second Peptide Tag Pairs

Conjugation of the first and second peptide tag/binding partner/reactive pairs may be carried out overnight at 4° C. Alternatively, the conjugation reaction may be conducted at room temperature for 3-4 hours as coupling speed is expected to be increased at room temperature. The optimal first and second binding partner ratio for a given coupling reaction is dependent on the size of each binding partner. For example, a 1:1.5 molar ratio of VLP monomer to antigen may be sufficient for smaller antigens (˜20 kDa), whereas, a 1:1 mass ratio may be sufficient for larger antigens (>100 kDa) in combination with the same VLP monomer. However, both ratios result in excess antigen (the smaller binding partner). Any excess antigen can be removed by e.g. size exclusion chromatography (SEC) or by dialysis. Dialysis may be more suitable for smaller antigens as it is not as efficient as SEC. Alternatively, the ratio of VLP/particle to antigen may be optimised so that all of the antigen is conjugated and downstream purification is therefore not required. A suitable final protein concentration of approximately 1 mg/ml is optimal for conjugation reactions, as lower concentrations can reduce the reaction speed. A wide range of buffers near neutral pH are compatible with coupling/conjugation. A standard choice of conjugation buffer is TBS (20 mM Tris and 150 mM NaCl, pH 7.4). In some circumstances the addition of a 10× stock of citrate buffer (40 mM Na₂HPO₄, 200 mM sodium citrate, pH 6.2) may be used as described by Brune et al. Sci Rep. (2016).

### Pharmaceutical Composition and Use

The compositions of the invention may be incorporated into a vaccine or immunogenic composition. Suitably, a vaccine or immunogenic composition will comprise particles of the invention in an immunogenic dose.

A pharmaceutical composition may comprise a particle or immunogenic composition in accordance with the invention provided with a pharmaceutically acceptable carrier. Suitable carriers are well known to those skilled in the art. In one embodiment a pharmaceutical composition comprises a buffer, excipient or carrier. Suitably a pharmaceutical composition may comprise suitable excipients and formulations to maintain stability of the composition. Suitably the formulation may comprise an adjuvant. In one embodiment, the formulation may comprise AddaVax™ or a similar squalene-based oil-in-water nano-emulsion with a formulation similar to MF59®. Other suitable adjuvants include liposome-based adjuvants such as Matrix M and AS01. Other suitable adjuvants include aluminium-based formulations such as Alhydrogel®. In one embodiment the formulation may comprise EDTA, for example at a concentration of 5 mM. Suitable excipients or formulations may depend on the properties of the particle or immunogenic composition; for example, the choice of expression system may affect the stability, glycosylation or folding of the proteins of the composition, which may in turn affect the optimal formulation of the composition. Methods of determination of a suitable excipient, formulation or adjuvant will be known to those skilled in the art.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

“Recombinant” as used herein to describe a polynucleotide means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

Unless specifically stated, a process comprising steps may be performed in any suitable order. Thus steps can be performed in any appropriate order.

Sequence identity between polypeptide sequences is preferably determined by pairwise alignment algorithm using the Needleman-Wunsch global alignment algorithm (Needleman and Wunsch 1970), using default parameters (e.g. with Gap opening penalty=10.0, and with Gap extension penalty=0.5, using the EBLOSUM62 scoring matrix). This algorithm is conveniently implemented in the needle tool in the EMBOSS package (Rice, Longden and Bleasby 2000). Sequence identity should be calculated over the entire length of the polypeptide sequence of the invention.

Any homologues of components mentioned herein are typically a functional homologue and are typically at least 40% homologous to the relevant region of the protein. Homology can be measured using known methods. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/).

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

A variant polypeptide comprises (or consists of) sequence which has at least 40% identity to the native protein. In preferred embodiments, a variant sequence may be at least 55%, 65%, 70%, 75%, 80%, 85%, 90% and more preferably at least 95%, 97% or 99% homologous to a particular region of the native protein over at least 20, preferably at least 30, for instance at least 40, 60, 100, 200, 300, 400 or more contiguous amino acids, or even over the entire sequence of the variant. Alternatively, the variant sequence may be at least 55%, 65%, 70%, 75%, 80%, 85%, 90% and more preferably at least 95%, 97% or 99% homologous to full-length native protein. Typically the variant sequence differs from the relevant region of the native protein by at least, or less than, 2, 5, 10, 20, 40, 50 or 60 mutations (each of which can be substitutions, insertions or deletions). A variant sequence of the invention may have a percentage identity with a particular region of the full-length native protein which is the same as any of the specific percentage homology values (i.e. it may have at least 40%, 55%, 80% or 90% and more preferably at least 95%, 97% or 99% identity) across any of the lengths of sequence mentioned above.

Variants of the protein also include truncations. Any truncation may be used so long as the variant is still functional. Truncations will typically be made to remove sequences that are non-essential for activity/function, in particular the formation of an isopeptide bond, and/or do not affect conformation of the folded protein, in particular folding of any immunogenic sites. Truncations may also be selected to improve ease of production of the components. Appropriate truncations can routinely be identified by systematic truncation of sequences of varying length from the N- or C-terminus.

Variants of the native protein further include mutants which have one or more, for example, 2, 3, 4, 5 to 10, 10 to 20, 20 to 40 or more, amino acid insertions, substitutions or deletions with respect to a particular region of the native protein. Deletions and insertions are made preferably outside of the antigenic areas. Insertions are typically made at the N- or C-terminal ends of a sequence derived from the native protein, for example for the purposes of recombinant expression. Substitutions are also typically made in regions that are non-essential for activity/function and/or do not affect conformation of the folded protein. Such substitutions may be made to improve solubility or other characteristics of the protein. Substitutions may be made in order to increase the stability of the protein.

Substitutions preferably introduce one or more conservative changes, which replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid. Conservative amino acid changes are well known in the art.

A derivative is an entity that arises or is made from a parent entity by replacement of some part of the parent entity.

### EXAMPLES

### Example 1

### Generation of Exemplary Multimer—VLP Composition (HCMV Pentamer-HBsAg VLP)

HCMV pentamer was expressed transiently in Expi293F cells using ExpiFectamine™ 293 transfection reagents (ThermoFisher Scientific) and 5 separate plasmids encoding the sequences below. The HCMV pentamer described below is approx. 162 kDa without glycosylation (including tags and linkers but excluding signal peptides).

### Nucleotide Sequences

The HCMV pentamer sequences expressed represent native sequences from the Merlin strain (GenBank: AY446894.2; low-passage (i.e. attenuated) HCMV strain) (including introns), except for two introduced mutations (one in gH, one in UL128) described in the relevant passages below.

gH-SpyTag-His Nucleotide Sequence (SEQ ID NO. 12)

In this sequence (SEQ ID NO:12), a silent mutation C>A at position 1146 was introduced for GeneArt® synthesis, as the native sequence CACCTGC around this nucleotide was flagged up as possibly problematic. The construct comprises: Signal peptide (nt 1-69), Ectodomain (nt 70-2151), transmembrane domain (truncated) (nt 2152-2157), (the signal peptide, ectodomain and transmembrane domain (truncated) together being represented by SEQ ID NO:13), Linker (nt 2158-2175; SEQ ID NO:14), SpyTag (nt 2176-2214; SEQ ID NO:15), 6x His tag (nt 2215-2232), Stop codon (nt 2233-2235). Nucleotides 1 to 2157 (SEQ ID NO:13) represent the gH coding sequence.

### gL Nucleotide Sequence (SEQ ID NO. 16)

In this sequence: Signal peptide (nt 1-90), Ectodomain (nt 91-834), Stop codon (nt 835-837).

### UL130-C-Tag Nucleotide Sequence (SEQ ID NO. 17)

In this sequence: Signal peptide (nt 1-75), Ectodomain (nt 76-642), Linker (nt 643-687), C-tag (nt 688-699), Stop codon (nt 700-702).

### UL128 Nucleotide Sequence (SEQ ID NO. 20) (Includes the 2 Introns Present in the Native Sequence)

In this sequence: Signal peptide (nt 1-81), Introns: nt 165-287, nt 423-542, Ectodomain exons (nt 82-164, nt 288-422, nt 543-756), Stop codon (nt 757-759).

A T>C mutation was introduced at nucleotide 634. The T634 nucleotide was mentioned in the GenBank file as causing premature termination of UL128 in the Merlin strain, and we therefore used annotations from a different strain (GenBank: GQ396662.1, strain HAN38) to inform which base to substitute to in order to revert to expression of the full-length protein.

### UL131A Nucleotide Sequence (SEQ ID NO. 21) (Includes the Intron Present in the Native Sequence)

In this sequence: Signal peptide (nt 1-54), Intron (nt 237-344, Ectodomain exons (nt 55-236, nt 345-495), Stop codon (nt 496-498).

### SpyCatcher-HBsAg Nucleotide Sequence (SEQ ID NO. 22)

In this sequence: SpyCatcherDeltaN1 (nt 1-276), flexible Linker (nt 277-303), PVTN linker (nt 304-315), HBsAg (nt 316-993), C-tag (nt 994-1005), Stop codon (nt 1006-1008).

### Amino Acid Sequences

Expression of the above nucleotide sequences is predicted to result in the below amino acid sequences.

gH-SpyTag-His Amino Acid Sequence (SEQ ID NO. 27)

Predicted mass 81.852 kDa (without signal peptide), 84.364 kDa (including signal peptide). In this sequence: Signal peptide (aa 1-23), Ectodomain (aa 24-717), Transmembrane domain (truncated) (aa 718-719)) (the signal peptide, ectodomain and transmembrane domain (truncated) together represented by SEQ ID NO:28), Linker (aa 720-725; SEQ ID NO:29), SpyTag (aa 726-738; SEQ ID NO:30), 6x His tag (aa 739-744). Amino acid residues 1-719 represent the native Merlin strain gH amino acid sequence with truncated TM domain (SEQ ID NO:28).

### gL Amino Acid Sequence (SEQ ID NO:31)

Predicted mass 27.522 kDa (without signal peptide), 30.815 kDa (including signal peptide). In this sequence: Signal peptide (aa 1-30), Ectodomain (aa 31-278). Amino acid residues 1-278 represent the native Merlin strain gL amino acid sequence.

### UL130-C-Tag Amino Acid Sequence (SEQ ID NO:32)

Predicted mass 23.167 kDa (without signal peptide), 26.081 kDa (including signal peptide). In this sequence: Signal peptide (aa 1-25), Ectodomain (aa 26-214), (signal peptide and ectodomain together represented by SEQ ID NO:33), Linker (aa 215-229; SEQ ID NO:34), C-tag (aa 230-233).

Amino acid residues 1-214 represent the native Merlin strain UL130 amino acid sequence.

### UL128 Amino Acids Sequence (SEQ ID NO:35)

Predicted mass 16.659 kDa (without signal peptide), 19.717 kDa (including signal peptide). In this sequence: Signal peptide (aa 1-27), Ectodomain (aa 28-171). Amino acid residues 1-171 represent the native Merlin strain UL128 amino acid sequence.

### UL131A Amino Acid Sequence (SEQ ID NO:36)

Predicted mass 12.985 kDa (without signal peptide), 14.989 kDa (including signal peptide). In the above sequence: Signal peptide (aa 1-18), Ectodomain (aa 19-129). Amino acid residues 1-129 represent the native Merlin strain UL131A amino acid sequence.

### SpyCatcher-HBsAg Amino Acid Sequence (SEQ ID NO:37)

Predicted mass 36.824 kDa including tags and linkers.

In this sequence: SpyCatcherDeltaN1 (aa 1-92; SEQ ID NO:38), Flexible Linker (aa 93-101; SEQ ID NO:39), PVTN linker (aa 102-105; SEQ ID NO:40), HBsAg (aa 106-331; SEQ ID NO:41), C-tag (aa 332-335).

### Purification of the Pentamer

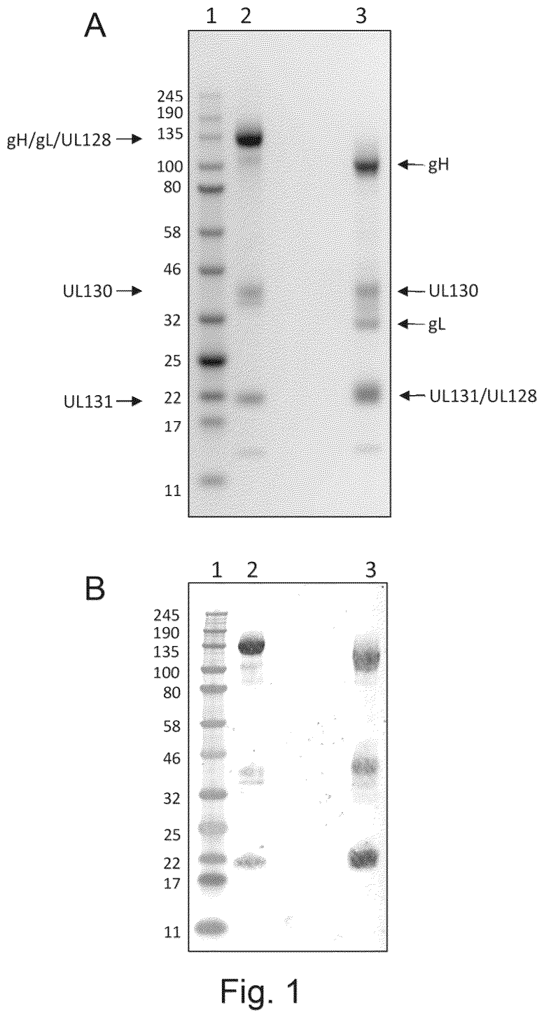

Pentamer-SpyTag was expressed in EXPI293F cells and was secreted into the supernatant (due to the deletion of (a portion of) the TM domain from the gH subunit). Initial attempts to use affinity purification to purify the HCMV pentamer relied on the expression of the gH subunit with a C-tag, but this resulted in the isolation of gH/gL hetero homodimers as well as the pentamer. In an alternative strategy a C-tag was added to the UL130 subunit (SEQ ID NO:17 (nucleotide) and SEQ ID NO:32 (amino acid)) which permitted purification of the pentamer from the supernatant using C-tag affinity purification (ThermoFisher) and size exclusion chromatography. The pentamer appeared as expected under non-reducing and reducing conditions when analysed by SDS-PAGE (FIG. 1A) and reacted with anti-HCMV pentamer antibodies (Native Antigen Company (AbCMV2450)) (FIG. 1B), with only minor contaminants observed at ˜14 kDa.

### Purification of the HBsAg VLP Monomer

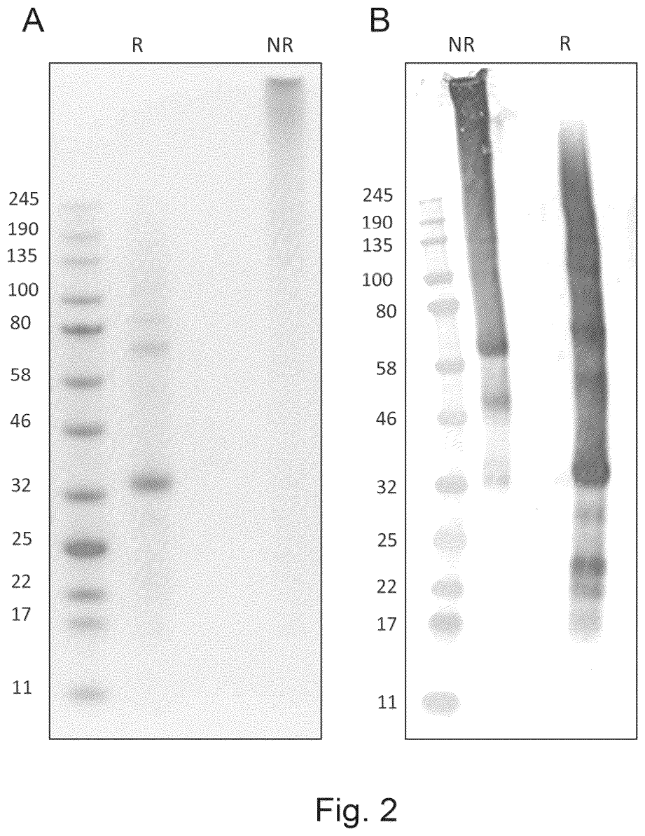

SpyCatcher-HBsAg was expressed in Pichia pastoris and purified from the cell homogenate. Under reducing conditions on an SDS-PAGE gel the predominant protein band corresponded to the expected size of the monomer (approx. 37 kDa) with further larger bands indicating the presence of oligomeric species, indicating that good cross-linking of the particle had occurred (FIG. 2A, lane ‘R’). Under non-reducing conditions (lane ‘NR’) the material predominantly remained at the top of the gel with some smearing, which indicates that the VLP particle was well formed and therefore too large to fully migrate into the gel (FIG. 2A). Both non-reduced and reduced SpyCatcher-HBsAg reacted strongly with a mouse anti-HBsAg monoclonal antibody (obtained from Bio-Rad (MCA4658)) (FIG. 2B), indicating that the presence of SpyCatcher did not negatively affect the reactive epitope.

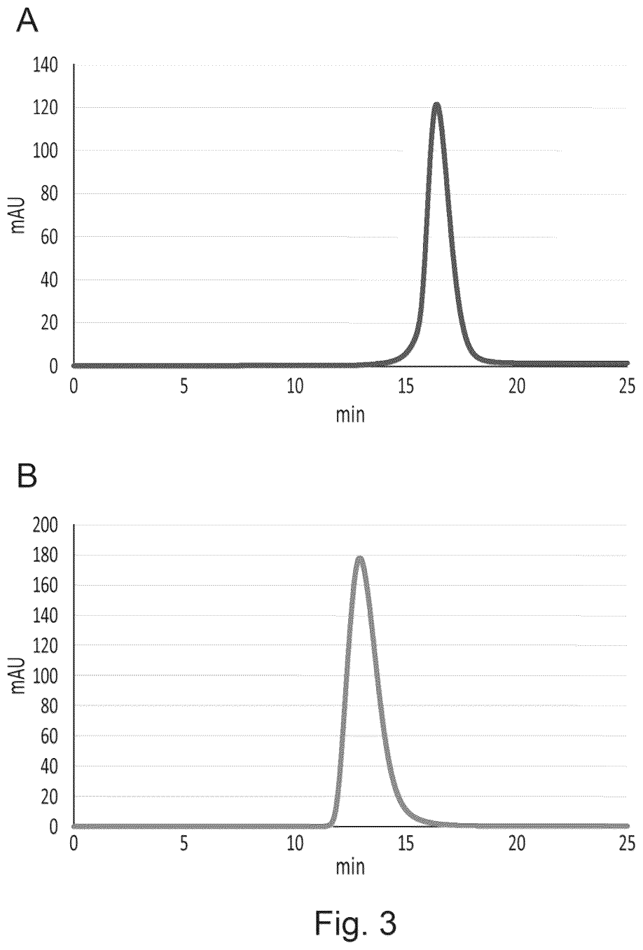

Both HCMV pentamer-SpyTag and SpyCatcher-HBsAg eluted as single peaks as assessed by HPLC size exclusion analysis on an s200increase 3.2/300 column (FIG. 3A-B). HCMV pentamer-SpyTag eluted at ˜400 kDa (FIG. 3A) which is larger than expected. However, this can be explained by the structure of the pentamer not being spherical which is known to alter the retention times of proteins during size exclusion chromatography. SpyCatcher-HBsAg eluted in the void volume of the column, which indicates that the particle is properly formed with no monomer detectable in the solution (FIG. 3B).

### Antigen-VLP Conjugation

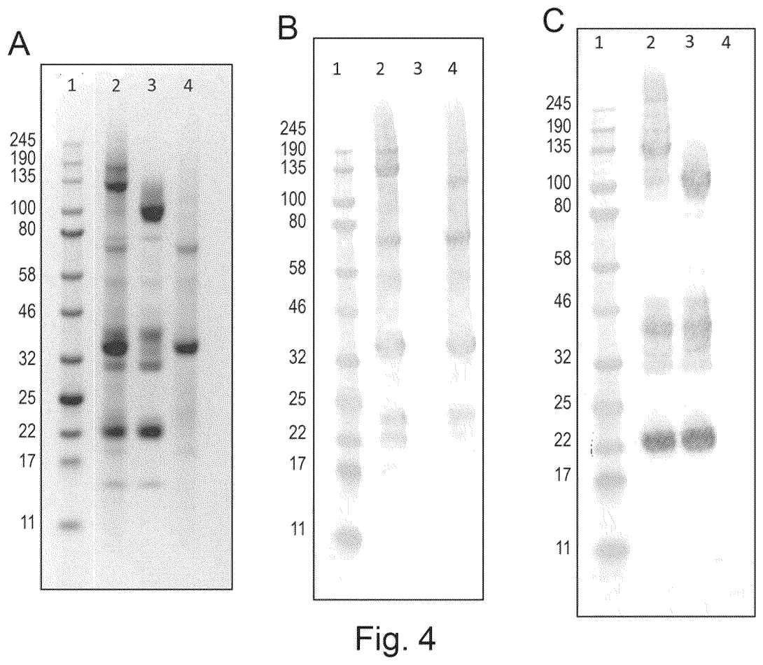

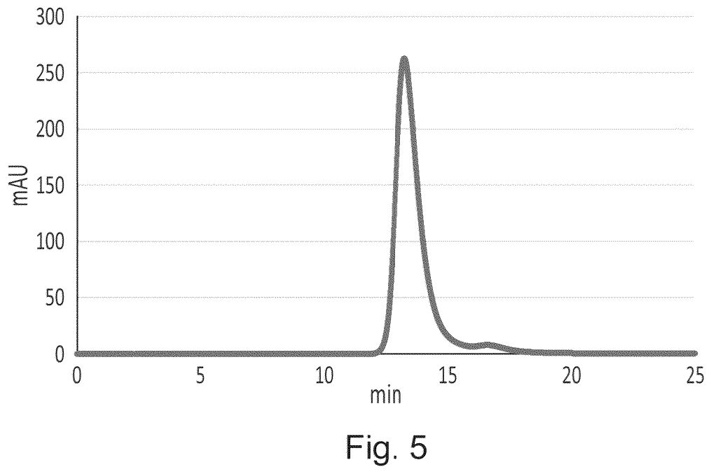

HCMV pentamer-SpyTag was conjugated to SpyCatcher-HBsAg overnight at 4° C. resulting in an HBsAg VLP coated with HCMV-pentamer. A buffer containing Tris buffered saline (TBS; 20 mM Tris and 150 mM NaCl, pH 7.4) supplemented with 5 mM EDTA was used for conjugation. The conjugation was monitored using SDS-PAGE and Western-blot analysis as well as HPLC. When the conjugation reaction was compared to either pentamer-SpyTag or SpyCatcher-HBsAg alone there was the presence of a new band at ˜130 kDa under reducing conditions (FIG. 4A, lane 2) which was reactive with both monoclonal anti-HBsAg (FIG. 4B) and polyclonal anti-HCMV pentamer (FIG. 4C) antibodies, indicating it contained conjugated HBsAg-gH at least. When analysed by HPLC size exclusion chromatography 97% eluted in the main peak corresponding to the predicted size of conjugated HCMV pentamer-HBsAg monomer (FIG. 5).

### Example 2

### In Vivo Testing of HCMV-SpyTag--SpyCatcher-HBsAg VLP (Adjuvanted)

The conjugated HCMV pentamer-HBsAg VLP, as well as unconjugated HCMV pentamer-SpyTag, were used in an immunisation schedule using BALB/c mice to (i) confirm the immunogenicity of the HCMV pentamer-SpyTag produced and (ii) to compare the immunogenicity of the unconjugated HCMV pentamer-SpyTag versus conjugated HCMV pentamer-HBsAg VLP.

A Prime-Boost-Boost schedule with 3 week intervals was used as follows:

Day 0: immunisation (prime); Day 20: tail bleed; Day 21: immunisation (boost 1); Day 41: tail bleed; Day 42: immunisation (boost 2); Day 63: cardiac bleed.

The immunised groups were as follows. For each group n=10:
1) 1 μg HCMV pentamer-SpyTag in AddaVax™ (Invivogen)
2) 1 μg HCMV pentamer-SpyTag--SpyCatcher-HBsAg VLP (1 μg of pentamer equivalent) in Adda Vax™
3) SpyCatcher-HBsAg VLP (normalised to the amount of SpyCatcher-HBsAg in group 2) in Adda Vax™
4) 0.1 μg HCMV pentamer-SpyTag in Adda Vax™
5) 0.1 μg HCMV pentamer-SpyTag--SpyCatcher-HBsAg VLP (0.1 μg of pentamer equivalent) in AddaVax™
6) TBS (20 mM Tris and 150 mM NaCl, pH 7.4)

Adda Vax™ is a squalene-based oil-in-water nano-emulsion with a formulation similar to MF59® that has been licensed in Europe for adjuvanted flu vaccines. Squalene oil-in-water emulsions are known to elicit both cellular (Th1) and humoral (Th2) immune responses. Other suitable adjuvants will be known to those skilled in the art.

Immunogenicity was assessed using ELISA. A standardised ELISA against HCMV pentamer was used to determine the titre of the antisera raised in each group. Plates were coated overnight with 5 μg/ml pentamer (without SpyTag), 50 μL/well; washed; blocked with milk for one hour; washed; mouse sera (at an appropriate dilution in PBS) applied for 1 hour; washed; goat anti-mouse-Alkaline Phosphatase antibody (1:10,000) applied for one hour; washed; developed. Both unconjugated (Groups 1 and 4) and conjugated HCMV pentamer-HBsAg (Groups 2 and 5) at different doses were included to permit the comparison of immunogenicity between the conjugated HCMV pentamer-HBsAg VLP vaccine and unconjugated HCMV pentamer-SpyTag, which allows extrapolation to other HCMV pentamer vaccines (e.g. soluble pentamer). Groups 3 and 6 represent negative controls.

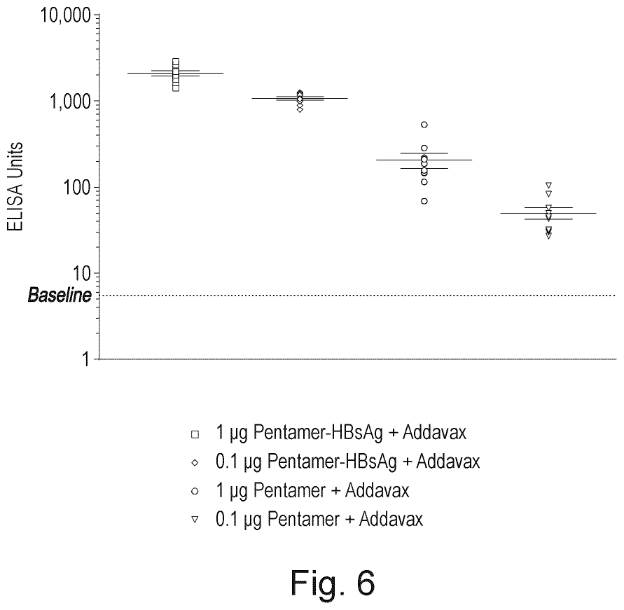

At each time point, OD values for the samples were read at appropriate dilutions, and ELISA Units determined using a standard curve run on each plate. Data showing the results for groups 1, 2, 4 and 5 post-prime is shown in FIG. 6. HCMV pentamer-HBsAg immunised mice show substantially stronger serum IgG antibody responses using both 1 μg and 0.1 μg doses, in comparison to mice immunised with 1 μg or 0.1 μg doses of unconjugated HCMV pentamer. ELISA units for groups 3 and 6 provided the baseline for this assay, also shown in FIG. 6.

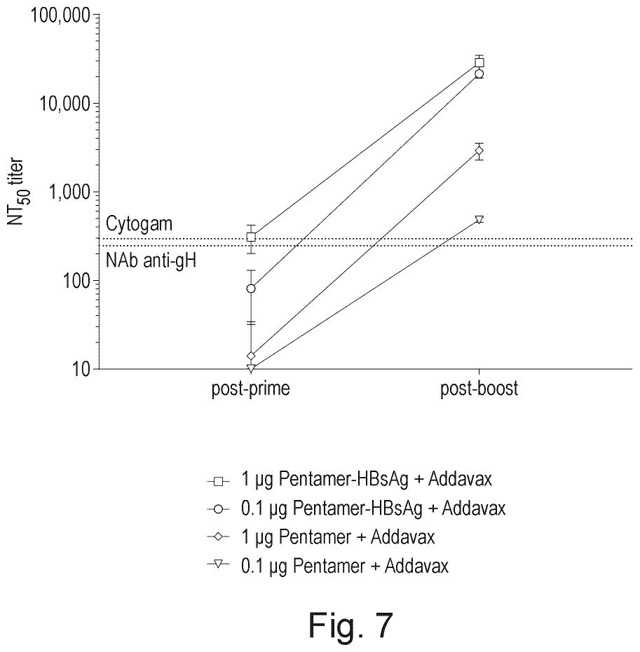

The functional activity of the antibodies raised was investigated using a microneutralisation assay based upon Wang et al. (Vaccine 33 (2015) 7254-7261; DOI:10.1016/j.vaccine.2015.10.110). Neutralising titres for groups 1, 2, 4 and 5 are shown in FIG. 7. The sera from mice immunised with pentamer-HBsAg VLP are substantially more neutralising than those of mice immunised with pentamer-SpyTag protein alone.

### Example 3

### Stable Construct Sequences

Two stable constructs (adapted from Hofmann et al., (2015) Biotech and Bioeng, 112(12):2505-2515) were optimised for CHO expression of components of the HCMV pentamer-SpyTag. Introns were removed from the HCMV pentamer sequences but the signal sequences were retained.

HCMV gH-SpyTag/gL Stable Expression Construct

Stable vector construct HCMV-gH-(GSG) 2-SpyTag-His-IRES-gL was designed to comprise the gH-SpyTag-His component (SEQ ID NO:42) and the gL component (SEQ ID NO:43), respectively upstream and downstream of the EV71 IRES. The coding sequences used in this construct are described below.

### Nucleotide Sequences

gH-(GSG) 2-SpyTag-His (Without Introns) Inserted Upstream of EV71 IRES (SEQ ID NO:42)

In this sequence: Signal peptide (nt 1-69), Ectodomain (nt 70-2151), Truncated transmembrane domain (nt 2152-2157), (GSG) 2 linker (nt 2158-2175), SpyTag (nt 2176-2214), His-tag (nt 2215-2232), Stop codon (nt 2233-2235).

### gL (Without Introns) Inserted Downstream of EV71 IRES (SEQ ID NO:43)

In this sequence: Signal peptide (nt 1-90), Ectodomain (nt 91-834), Stop codon (nt 835-837)

### HCMV UL128/UL130/UL131A Stable Expression Construct

Stable construct HCMV-UL128-IRES-UL130-(G4S) 3-C-tag-IRES-UL131A was designed to comprise the UL128 component (SEQ ID NO:44), the UL130 component (SEQ ID NO:45) and the UL131A component (SEQ ID NO:46). The UL130 component was inserted after the first EV71 IRES of the plasmid and the UL131A component was inserted after the second EV71 IRES. The coding sequences used in this construct are described below.

### Nucleotide Sequences

### UL128 (Without Introns) (SEQ ID NO:44)

In this sequence: Signal peptide (nt 1-81), Ectodomain (nt 82-513), Stop codon (nt 514-516).

### UL130-(G4S)3-C-tag (Without Introns) (SEQ ID NO:45)

In this sequence: Signal peptide (nt 1-75), Ectodomain (nt 76-642), (G4S) 3 linker (nt 643-687), Ctag (nt 688-699), Stop codon (nt 700-702).

### UL131A (Without Introns) (SEQ ID NO:46)

In this sequence: Signal peptide (nt 1-54), Ectodomain (nt 55-387), Stop codon (nt 388-390).

### Example 4

### In Vivo Testing of HCMV-SpyTag--SpyCatcher-HBsAg VLP (Unadjuvanted)

The conjugated HCMV pentamer-HBsAg VLP, as well as unconjugated HCMV pentamer-SpyTag, were used in an immunisation schedule using BALB/c mice to further study the immunogenicity of the conjugated pentamer-HBsAg VLP versus unconjugated pentamer-SpyTag protein.

A Prime-Boost-Boost schedule with 3 week intervals was used as follows:

Day 0: immunisation (prime); Day 20: tail bleed; Day 21: immunisation (boost 1); Day 41: tail bleed; Day 42: immunisation (boost 2); Day 63: cardiac bleed.

The immunised groups were as follows. For each group n=10:1)
1) 1 μg HCMV pentamer-SpyTag unadjuvanted
2) 1 μg HCMV pentamer-SpyTag--SpyCatcher-HBsAg VLP (1 μg of pentamer equivalent) unadjuvanted
3) 0.1 μg HCMV pentamer-SpyTag--SpyCatcher-HBsAg VLP (0.1 μg of pentamer equivalent) unadjuvanted

Immunogenicity was assessed using ELISA. A standardised ELISA against HCMV pentamer was used to determine the titre of the antisera raised in each group. Plates were coated overnight with 5 μg/ml pentamer (without SpyTag), 50 μL/well; washed; blocked with milk for one hour; washed; mouse sera (at an appropriate dilution in PBS) applied for 1 hour; washed; goat anti-mouse-Alkaline Phosphatase antibody (1:10,000) applied for one hour; washed; developed.

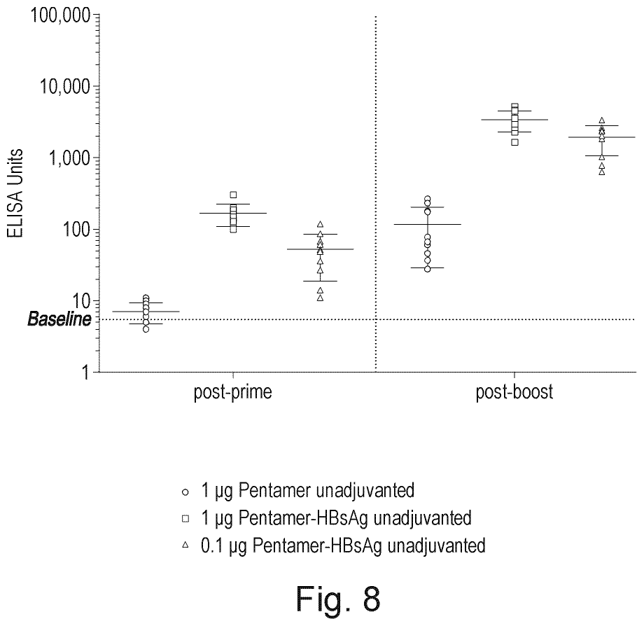

At each timepoint, OD values for the samples were read at appropriate dilutions, and ELISA Units determined using a standard curve ran on each plate. Post-prime and post-boost data is shown in FIG. 8. HCMV pentamer-HBsAg immunised mice show substantially stronger serum IgG antibody responses using both 1 μg and 0.1 μg doses, in comparison to mice immunised with 1 μg of HCMV pentamer alone as soluble protein.

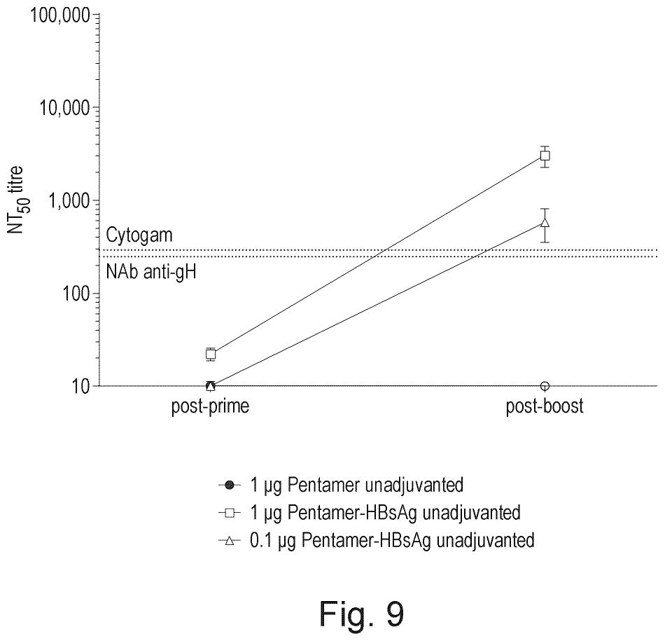

The functional activity of the antibodies raised was investigated using a microneutralisation assay based upon Wang et al. (2015). Post-prime and post-boost neutralising titres are shown in FIG. 9. The sera from mice immunised with unadjuvanted pentamer-HBsAg VLP are substantially more neutralising than those of mice immunised with unadjuvanted pentamer-SpyTag protein alone.

### Example 5

### Expression and Purification of RSV-F-SpyTag

The sequence from antigen RSV-F Sc9-10 DS-Cav1 A149C Y458C was fused to SpyTag to generate RSV-F-SpyTag, and was expressed by transiently transfecting ExpiCHO™ cells with the nucleotide sequence SEQ ID NO:47 in plasmid pcDNA3.4, using ExpiCHO™ Expression System Kit and ExpiFectamine™ transfection reagents (ThermoFisher Scientific).

RSV-F Sc9-10 DS-Cav1 A149C Y458C (National Institutes of Health) is a variant of the Respiratory Syncytial Virus Fusion protein (pre-fusion RSV-F) as described by Joyce et al. (2016) (Iterative structure-based improvement of a respiratory syncytial virus fusion glycoprotein vaccine. Nat Struct Mol Biol. 2016 September; 23(9):811-820). This variant is a pre-fusion-stabilised form of the fusion (F) glycoprotein with genetically-linked F subunits, fusion peptide deleted, T4 fibritin trimerisation motif (foldon domain), and interprotomer movements stabilised by an additional interprotomer disulfide bond (A149C Y458C).

### Nucleotide Sequences

### RSV-F-SpyTag-Ctag Nucleotide Sequence (SEQ ID NO:47)

The original sequence of Sc9-10 DS-Cav1 A149C Y458C was modified at the 3′ end by the deletion of the thrombin site, 6x His-tag and Strep-tag® II. These deleted domains were replaced with a linker-SpyTag-C-tag sequence, to produce a 1587 nt cassette (SEQ ID NO:47) comprising Sc9-10 DS-Cav1 A149C Y458C (nt 1-1515, including signal peptide (nt 1-75), and T4 fibritin foldon domain (nt 1435-1515)), (GSG) 2 linker (nt 1516-1533; SEQ ID NO:14), SpyTag (nt 1534-1572; SEQ ID NO:15), C-tag (nt 1573-1584) and Stop codon (nt 1585-1587). The Sc9-10 DS-Cav1 A149C Y458C nucleotide sequence excluding the linker, SpyTag, C-tag and Stop codon is encompassed by SEQ ID NO:48 The Sc9-10 DS-Cav1 A149C Y458C nucleotide acid sequence excluding the signal peptide, linker, SpyTag or C-tag is encompassed by SEQ ID NO:49.

### Amino Acid Sequences

Expression of nucleotide sequence SEQ ID NO:47 was predicted to result in an RSV-F-SpyTag-Ctag amino acid sequence (SEQ ID NO:50) with the following domains: Sc9-10 DS-Cav1 A149C Y458C ((aa 1-505, including signal peptide (aa 1-25) and foldon domain (aa 479-505)), linker (aa 506-511; SEQ ID NO:29), SpyTag (aa 512-524; SEQ ID NO:30), C-tag (aa 525-528). The predicted mass of the protein was 57.9 kDa with the signal peptide, 55.3 kDa without the signal peptide. The Sc9-10 DS-Cav1 A149C Y458C amino acid sequence excluding the linker, SpyTag or C-tag is encompassed by SEQ ID NO:51. The Sc9-10 DS-Cav1 A149C

Y458C amino acid sequence excluding the signal peptide, linker, SpyTag or C-tag is encompassed by SEQ ID NO:52.

### Purification of RSV-F-SpyTag

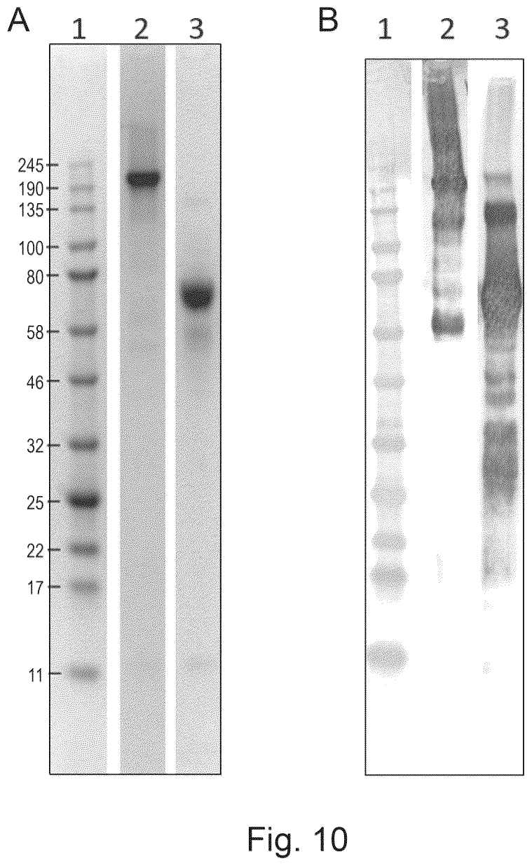

The RSF-F-SpyTag antigen was secreted from the cells and purified from the supernatant using C-tag affinity purification and size exclusion chromatography. RSV-F-SpyTag appeared as expected under non-reducing and reducing conditions when analysed by SDS-PAGE (FIG. 10A) and reacted with anti-RSV-F [2F7] monoclonal antibody (ab43812; Abcam) (FIG. 10B).

### Purification of the HBsAg VLP Monomer

SpyCatcher-HBsAg (VLP monomer) was prepared and purified as described in Example 1 above, see also FIG. 2.

### Conjugation of RSV-F-SpyTag to SpyCatcher-HBsAg

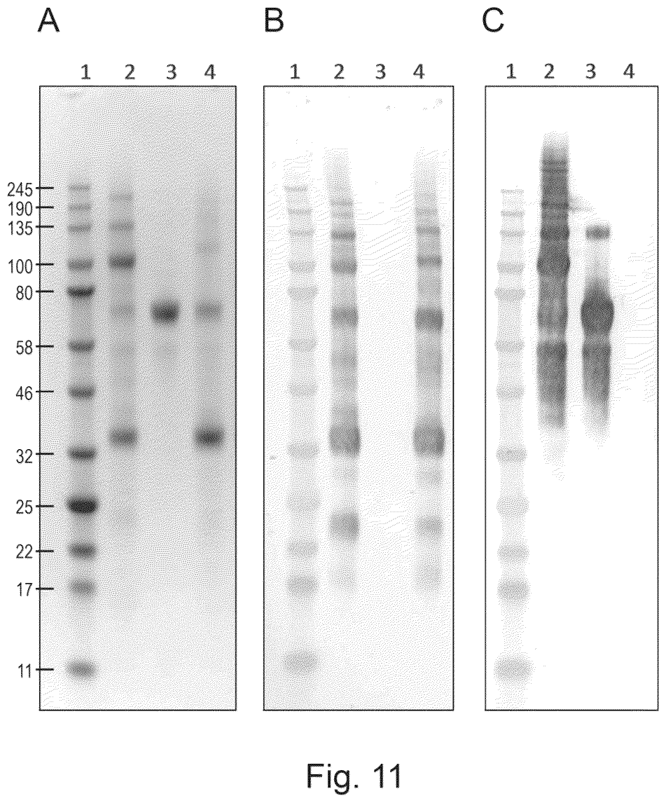

RSV-F-SpyTag was conjugated to SpyCatcher-HBsAg overnight at 4° C. resulting in a HBsAg VLP coated with RSV-F trimer (RSV-F-SpyTag--SpyCatcher-HBsAg). A buffer containing Tris buffered saline (TBS; 20 mM Tris and 150 mM NaCl, pH 7.4) was used for conjugation. The conjugation was monitored using SDS-PAGE and Western-blot analysis (FIG. 11). When the conjugation reaction was compared to either RSV-F-SpyTag or SpyCatcher-HBsAg alone there was the presence of a new band at ˜105 kDa (lane 2) under reducing conditions (FIG. 11A) which was reactive with both anti-HBsAg monoclonal antibody (MCA4658, Bio-Rad) (FIG. 11B) and anti-RSV-F [2F7] monoclonal antibody (ab43812; Abcam) (FIG. 11C), indicating it contained conjugated RSV-F-SpyTag--SpyCatcher-HBsAg.

### Example 6

### Immunogenicity of Conjugated RSV-F-SpyTag--SpyCatcher-HBsAg

An immunisation schedule was designed using BALB/c mice to confirm the immunogenicity of the produced RSV-F antigen and to compare the immunogenicity of the conjugated RSV-F-SpyTag--SpyCatcher-HBsAg VLP versus unconjugated RSV-F-SpyTag protein. The groups were dosed based on the amount of RSV-F-SpyTag in the sample, and a Prime-Boost schedule with 3 weeks interval was selected with the final time point 2 weeks after the boost immunisation.

Post-prime mice immunised with RSV-F-SpyTag--SpyCatcher-HBsAg show substantially stronger serum IgG antibody responses compared to mice immunised with RSV-F-SpyTag protein alone irrespective of whether the vaccines were unadjuvanted (FIG. 6) or formulated with Addavax™ (FIG. 6).

Table of sequences

| | Nucleotide | Amino acid |
|---|---|---|
| Sequence | sequence | sequence |
| Flexible linker 2 - (GSG)₂ | n/a | SEQ ID NO: 1 |
| Flexible linker 3 - (GSG)₃ | n/a | SEQ ID NO: 2 |
| Flexible linker 4 - (G4S)₁ | n/a | SEQ ID NO: 3 |
| Flexible linker 5 - (G4S)₃ | n/a | SEQ ID NO: 4 |
| Flexible linker 6 - (G4S)₄ | n/a | SEQ ID NO: 5 |
| Flexible linker 7 | n/a | SEQ ID NO: 6 |
| Flexible linker 8 | n/a | SEQ ID NO: 7 |
| Flexible linker 9 | n/a | SEQ ID NO: 8 |
| Rigid linker 1 | n/a | SEQ ID NO: 9 |
| Rigid linker 2 - (EAAAK)₃ | n/a | SEQ ID NO: 10 |
| Rigid linker 3 - (AP)₇ | n/a | SEQ ID NO: 11 |
| gH-SpyTag-His | SEQ ID NO: 12 | SEQ ID NO: 27 |
| gH with truncated transmembrane | SEQ ID NO: 13 | SEQ ID NO: 28 |
| domain | | |
| Linker from gH construct (2158- | SEQ ID NO: 14 | SEQ ID NO: 29 |
| 2175 bp) | | |
| SpyTag (2176-2214 bp) | SEQ ID NO: 15 | SEQ ID NO: 30 |
| gL | SEQ ID NO: 16 | SEQ ID NO: 31 |
| UL130-C-tag | SEQ ID NO: 17 | SEQ ID NO: 32 |
| U130 (signal sequence and | SEQ ID NO: 18 | SEQ ID NO: 33 |
| ectodomain) | | |
| Linker from UL130 construct | SEQ ID NO: 19 | SEQ ID NO: 34 |
| UL128 (includes the 2 introns) | SEQ ID NO: 20 | SEQ ID NO: 35 |
| UL131A (includes the intron) | SEQ ID NO: 21 | SEQ ID NO: 36 |
| SpyCatcher-HBsAg | SEQ ID NO: 22 | SEQ ID NO: 37 |
| SpyCatcherDeltaN1 | SEQ ID NO: 23 | SEQ ID NO: 38 |
| Flexible linker from SpyCatcher- | SEQ ID NO: 24 | SEQ ID NO: 39 |
| HBsAg | | |
| PVTN linker from SpyCatcher- | SEQ ID NO: 25 | SEQ ID NO: 40 |
| HBsAg | | |
| HBsAg | SEQ ID NO: 26 | SEQ ID NO: 41 |
| gH-SpyTag-His optimised for | SEQ ID NO: 42 | SEQ ID NO: 27 |
| CHO expression | | |
| gL optimised for CHO expression | SEQ ID NO: 43 | SEQ ID NO: 31 |
| UL128 optimised for CHO | SEQ ID NO: 44 | SEQ ID NO: 35 |
| expression | | |
| UL130 optimised for CHO | SEQ ID NO: 45 | SEQ ID NO: 32 |
| expression | | |
| UL131 optimised for CHO | SEQ ID NO: 46 | SEQ ID NO: 36 |
| expression | | |
| RSV-F-SpyTag-Ctag | SEQ ID NO: 47 | SEQ ID NO: 50 |
| Sc-9-10 DS-Cav1 A149C Y458C | SEQ ID NO: 48 | SEQ ID NO: 51 |
| (RSV-F) | | |
| Sc-9-10 DS-Cav1 A149C Y458C | SEQ ID NO: 49 | SEQ ID NO: 52 |
| without the signal peptide | | |
| RSV-F DS-Cav1-SpyTag-Ctag | SEQ ID NO: 53 | SEQ ID NO: 56 |
| RSV-F DS-Cav1 | SEQ ID NO: 54 | SEQ ID NO: 57 |
| RSV-F DS-Cav1 without the signal | SEQ ID NO: 55 | SEQ ID NO: 58 |
| peptide | | |
| gH with truncated transmembrane | SEQ ID NO: 59 | SEQ ID NO: 60 |
| domain without the signal peptide | | |

1. A composition comprising:
an antigenic component comprising a first peptide tag; and
a moiety comprising a second peptide tag,
wherein the antigenic component and the moiety are linked via an isopeptide bond between the first peptide tag and the second peptide tag, and wherein a molecular weight of the antigenic component is greater than 50 kDa.

2. The composition of claim 1, wherein the molecular weight of the antigenic component is greater than 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 300 kDa, or 400 kDa.

3. The composition of claim 1, wherein the antigenic component is a monomer or multimer.

4. The composition of claim 3, wherein the multimer is a dimer, trimer, tetramer, pentamer, hexamer, septamer, octamer, nonamer or decamer.

5. The composition of claim 1, wherein the moiety comprises a virus, a bacterium, a multimerisation scaffold for vaccination, a protein component which multimerises to form a virus-like particle (VLP), a viral structural protein, a multimerisation domain which forms nanoparticles, a synthetic nanoparticle, or a synthetic VLP.

6. The composition of claim 1, wherein the moiety comprises a Hepatitis B Surface Antigen (HBsAg).

7. The composition of claim 1, wherein the first peptide tag and the second peptide tag are selected from:
a SpyTag and SpyCatcher pair;
a SnoopTag or SnoopTagJr and SnoopCatcher pair;
a RrgATag, RrgATag2 or DogTag and RrgACatcher pair,
an IsopepTag Pilin-C pair;
an IsopepTag-N and Pilin-N pair;
a PsCsTag and PsCsCatcher pair; or
a SnoopTagJr and DogTag pair mediated by SnoopLigase.

8. The composition of claim 1, wherein the first peptide tag is a SpyTag.

9. The composition of claim 8, wherein SpyTag has the amino acid sequence set out in SEQ ID NO:30.

10. The composition of claim 8, wherein the SpyTag is attached via a linker.

11. The composition of claim 10, wherein the linker has the amino acid sequence set out in SEQ ID NO:29.

12. The composition of claim 1, wherein the moiety comprises a Hepatitis B Surface Antigen (HBsAg) and the second peptide tag is a SpyCatcher.

13. The composition of claim 12, wherein the SpyCatcher has the amino acid sequence set out in SEQ ID NO:38.

14. The composition of claim 12, wherein the SpyCatcher is attached to the HBsAg via a linker.

15. The composition of claim 14, wherein the linker has the amino acid sequence set out in SEQ ID NO:39.

16. An immunogenic composition or vaccine composition comprising the composition of claim 1.

17. A method of producing the composition of claim 1, the method comprising:
introducing a first nucleic acid encoding a first genetic fusion of a first protein to the first peptide tag into a first host cell;
incubating the first host cell under conditions for expressing the first genetic fusion;
introducing a second nucleic acid encoding a second genetic fusion of a second protein to the second peptide tag into a second host cell;
incubating the second host cell under conditions for expressing the second genetic fusion; and
incubating the first genetic fusion and the second genetic fusion under conditions for formation of an isopeptide bond between the first peptide tag and the second peptide tag to produce the composition.

18. The method of claim 17, wherein the first protein comprises the antigenic component and the second protein comprises the moiety.

19. A kit comprising a first immunogenic composition, wherein the first immunogenic composition comprises the composition of claim 1.

20. A kit comprising an immunogenic composition and a booster composition, wherein the immunogenic composition and/or the booster composition comprises the composition of claim 1.

21. A method of treating a subject, the method comprising administering to the subject the composition of claim 1.

22. The method of claim 21, wherein the method treats an infection or infectious agent in the subject.
